(19) United States
(12) Patent Application Publication
(10) Pub. No.: US 2023/0293839 A1
(43) Pub. Date: Sep. 21, 2023

(54) MEDICAL DEVICE FOR TREATING ESOPHAGEAL ATRESIA

(71) Applicant: BOSTON SCIENTIFIC SCIMED INC., MAPLE GROVE, MN (US)

(72) Inventors: RONALD L. LANCASTER, HOPKINTON, MA (US); MANDI ROUTON, MINNEAPOLIS, MN (US); JACOB HEINZ, MAPLE GROVE, MN (US); AIDEN FLANAGAN, Kilcolgan (IE)

(73) Assignee: BOSTON SCIENTIFIC SCIMED INC., MAPLE GROVE, MN (US)

(21) Appl. No.: 18/114,596

(22) Filed: Feb. 27, 2023

Related U.S. Application Data
(60) Provisional application No. 63/314,935, filed on Feb. 28, 2022.

(51) Int. Cl. A61M 16/04

(52) U.S. Cl. CPC A61M 16/044

(57) ABSTRACT

A medical device includes a handle, an elongate shaft extending distally from the handle, the elongate shaft configured to extend through a mouth of a patient and into an upper esophageal pouch of the patient, the elongate shaft having a distal end configured to engage the upper esophageal pouch, and a force absorbing member permitting the elongate shaft to translate axially relative to the handle. The force absorbing member is configured to exert a force along the elongate shaft to apply the force to the upper esophageal pouch. Another medical device includes a tubular member configured to extend through the mouth and into the upper esophageal pouch, a shaft extending distally from the tubular member, and an inflatable member fixedly attached to a distal end of the shaft. The inflatable member is configured to apply a force to the upper esophageal pouch in an inflated configuration.

### CROSS-REFERENCE TO RELATED APPLICATIONS

This application claims the benefit of U.S. Provisional Pat. Application Serial No. 63/314,935, filed Feb. 28, 2022, which is incorporated herein by reference.

### TECHNICAL FIELD

The present disclosure pertains to medical devices, and methods for using medical devices. More particularly, the present disclosure pertains to medical devices for treating esophageal atresia.

### BACKGROUND

Esophageal atresia is a condition where individuals are born with an incomplete esophagus which does not connect the throat to the stomach. The incomplete esophagus may form pouches having a closed end. Current treatment procedures, for example the Foker Technique, are very invasive and may cause extreme trauma. In some cases, individuals are chemically paralyzed to prevent motion for several weeks which may cause weight loss and muscle wasting, reduced brain size, and/or reduced bone density. Additionally, the Foker Technique requires a minimum body weight of 3.5 kilograms, which further limits its use in some situations such as a premature birth. The esophagus naturally grows in response to stretch as the individual grows, so the tissue “wants” to grow. The Foker Technique uses sutures which are surgically attached to both pouch ends of the esophagus and the stomach, passed through the skin on the patients back. The sutures are periodically pulled a small amount, inducing stretch on the esophagus causing growth over time. During that time, the individuals remain paralyzed to prevent suture and/or tissue rupture. Another shortcoming of this procedure is that the applied forces are uncontrolled and suture attachments to the pouches cause high stress concentrations leading to significant tissue morbidity that requires removal prior to anastomosis (surgical connection of the two ends to form a complete esophagus). In another alternative, magnets are implanted into each of the pouch ends of the esophagus and their attraction to each other applies force to the tissue and stretches the pouch ends of the esophagus towards each other over time. However, the force is non-linear and uncontrolled. Therefore, a minimum distance of 4 cm or less is required to have sufficient attraction and as the distance between the magnets decreases, the force applied to the tissue by the magnets increases, which may lead to rupture and/or additional tissue damage. Additionally, long-term sedation and/or immobilization, removal of mucous and/or saliva via suction, and alternative feeding procedures are still required, as is a surgical procedure to later remove the magnets and connect the pouch ends. As such, there is an ongoing need to provide alternative medical devices and procedures for treating esophageal atresia.

### SUMMARY

In one example, a medical device for treating esophageal atresia may comprise a handle; an elongate shaft extending distally from the handle, the elongate shaft configured to extend through a mouth of a patient and into an upper esophageal pouch of the patient, the elongate shaft having a distal end configured to engage a distal end of the upper esophageal pouch; and a force absorbing member disposed within the handle, the force absorbing member permitting the elongate shaft to translate axially relative to the handle, wherein the force absorbing member is configured to exert a force along the elongate shaft to apply the force to the distal end of the upper esophageal pouch.

In addition or alternatively to any example described herein, the medical device may further comprise an adjustment mechanism configured to adjust the force applied to the distal end of the upper esophageal pouch by the force absorbing member.

In addition or alternatively to any example described herein, the adjustment mechanism is configured to axially translate the force absorbing member within the handle.

In addition or alternatively to any example described herein, the medical device may further comprise a position sensor disposed within the handle, the position sensor being configured to detect an axial position of the elongate shaft relative to the handle.

In addition or alternatively to any example described herein, the elongate shaft includes an atraumatic distal head having a first outer diameter.

In addition or alternatively to any example described herein, the medical device may further comprise an elongate tubular member extending distally from the handle, the elongate tubular member having a second outer diameter less than the first outer diameter. At least a portion of the elongate shaft may be movably disposed within the elongate tubular member.

In addition or alternatively to any example described herein, the elongate shaft is fixedly secured relative to a distal end of the force absorbing member.

In addition or alternatively to any example described herein, the elongate shaft extends axially through the handle and engages with a longitudinal advancement-retraction mechanism disposed proximal of the handle.

In addition or alternatively to any example described herein, a medical device for treating esophageal atresia may comprise a handle including an axial translation mechanism; an elongate shaft extending distally from the handle, the elongate shaft configured to extend through a mouth of a patient and into an upper esophageal pouch of the patient, the elongate shaft having a distal end configured to engage a distal end of the upper esophageal pouch; and a spring disposed within the handle, the spring permitting the elongate shaft to translate axially relative to the handle in response to movements of the patient, wherein the spring is configured to exert a force along the elongate shaft to apply the force to the distal end of the upper esophageal pouch. The axial translation mechanism may be configured to adjust the force applied to the distal end of the upper esophageal pouch by the spring.

In addition or alternatively to any example described herein, the axial translation mechanism may include a knob disposed proximate a proximal end of the handle, and a threaded shaft extending distally from the knob within the handle. A distal portion of the threaded shaft may include a first flange abutting a proximal end of the spring and rotation of the knob relative to the handle translates the first flange axially within the handle.

In addition or alternatively to any example described herein, the elongate shaft extends axially through the knob and is movable with respect to the knob.

In addition or alternatively to any example described herein, a medical device for treating esophageal atresia may comprise an elongate tubular member configured to extend through a mouth of a patient and into an upper esophageal pouch of the patient; an elongate shaft extending distally from the elongate tubular member; and an inflatable member fixedly attached to a distal end of the elongate shaft, the inflatable member being configured to shift between a deflated configuration and an inflated configuration. The inflatable member may be configured to apply a force to a distal end of the upper esophageal pouch in the inflated configuration. The elongate shaft may include an inflation lumen in fluid communication with the inflatable member. The elongate tubular member may include an irrigation lumen extending therethrough and at least one suction lumen extending therethrough.

In addition or alternatively to any example described herein, the inflatable member is configured to expand in a longitudinal direction when shifting toward the inflated configuration while maintaining an outer diameter that is substantially constant.

In addition or alternatively to any example described herein, the medical device may further comprise a pressure regulator in fluid communication with the inflation lumen, the pressure regulator being responsive to sensed pressure within the inflatable member.

In addition or alternatively to any example described herein, a medical device for treating esophageal atresia may comprise a handle; an elongate shaft extending distally from the handle, the elongate shaft configured to extend through a mouth of a patient and into an upper esophageal pouch of the patient, the elongate shaft having a distal end configured to engage a distal end of the upper esophageal pouch; and a force absorbing member disposed within the handle, the force absorbing member permitting the elongate shaft to translate axially relative to the handle, wherein the force absorbing member is configured to exert a force along the elongate shaft to apply the force to the distal end of the upper esophageal pouch.

In addition or alternatively to any example described herein, the medical device may further comprise an adjustment mechanism configured to adjust the force applied to the distal end of the upper esophageal pouch by the force absorbing member.

In addition or alternatively to any example described herein, the adjustment mechanism is configured to axially translate the force absorbing member within the handle.

In addition or alternatively to any example described herein, the medical device may further comprise a position sensor disposed within the handle, the position sensor being configured to detect an axial position of the elongate shaft relative to the handle.

In addition or alternatively to any example described herein, the elongate shaft is fixedly secured relative to a distal end of the force absorbing member.

In addition or alternatively to any example described herein, the elongate shaft extends axially through the handle and engages with a longitudinal advancement-retraction mechanism disposed proximal of the handle.

In addition or alternatively to any example described herein, the force absorbing member is a spring.

In addition or alternatively to any example described herein, the adjustment mechanism may include a knob disposed proximate a proximal end of the handle; and a threaded shaft extending distally from the knob within the handle. A distal portion of the threaded shaft may include a first flange abutting a proximal end of the spring and rotation of the knob relative to the handle translates the first flange axially within the handle.

In addition or alternatively to any example described herein, the elongate shaft extends axially through the knob and is movable with respect to the knob.

In addition or alternatively to any example described herein, a medical device for treating esophageal atresia may comprise an elongate tubular member configured to extend through a mouth of a patient and into an upper esophageal pouch of the patient; an elongate shaft extending distally from the elongate tubular member; and an inflatable member fixedly attached to a distal end of the elongate shaft, the inflatable member being configured to shift between a deflated configuration and an inflated configuration. The inflatable member may be configured to apply a force to a distal end of the upper esophageal pouch in the inflated configuration. The elongate shaft may include an inflation lumen in fluid communication with the inflatable member. The elongate tubular member may include an irrigation lumen extending therethrough and at least one suction lumen extending therethrough.

In addition or alternatively to any example described herein, the inflatable member is spaced apart a fixed distance from a distal end of the elongate tubular member.

In addition or alternatively to any example described herein, the inflatable member is configured to expand in a longitudinal direction when shifting toward the inflated configuration while maintaining an outer diameter that is substantially constant.

In addition or alternatively to any example described herein, the medical device may further comprise a pressure regulator in fluid communication with the inflation lumen, the pressure regulator being responsive to sensed pressure within the inflatable member.

In addition or alternatively to any example described herein, at least a portion of the inflatable member is radiopaque.

In addition or alternatively to any example described herein, a method of treating esophageal atresia may comprise inserting a medical device for treating esophageal atresia into a mouth and esophagus of a patient. The medical device includes a handle including an adjustment mechanism, an elongate shaft extending distally from the handle, and a force absorbing member disposed within the handle. The elongate shaft has a distal end configured to engage a distal end of an upper esophageal pouch of the patient. The force absorbing member is configured to exert a force along the elongate shaft to apply the force to the distal end of the upper esophageal pouch. The method further includes positioning the distal end of the elongate shaft against the distal end of the upper esophageal pouch, fixing the medical device in place relative to the distal end of the upper esophageal pouch, and then actuating the adjustment mechanism to adjust the force applied to the distal end of the upper esophageal pouch by the force absorbing member. The force absorbing member is configured to permit the elongate shaft to translate axially relative to the handle while maintaining the force against the distal end of the upper esophageal pouch.

In addition or alternatively to any example described herein, the force is applied constantly to the distal end of the upper esophageal pouch.

In addition or alternatively to any example described herein, the force is applied cyclically the distal end of the upper esophageal pouch.

In addition or alternatively to any example described herein, the force is no more than 0.8 pounds of force.

In addition or alternatively to any example described herein, the method may further comprise, monitoring the force applied to the distal end of the upper esophageal pouch over time, monitoring axial translation of the elongate shaft over time, and actuating the adjustment mechanism to increase the force when the force drops below a predetermined force threshold or when axial translation of the elongate shaft reaches a predetermined translation threshold.

In addition or alternatively to any example described herein, a pressure sensor is disposed within the distal end of the elongate shaft, the pressure sensor being configured to measure the force applied to the distal end of the upper esophageal pouch.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description in connection with the accompanying drawings, in which:

FIG. 1 schematically illustrates aspects of esophageal atresia;

FIGS. 2-3 illustrate selected aspects of a medical device for treating esophageal atresia;

FIG. 4 illustrates selected aspects of an alternative configuration of the medical device of FIGS. 2-3;

FIG. 5 illustrates selected aspects of an alternative configuration of the medical device for treating esophageal atresia of FIGS. 2-3;

FIGS. 6-16 illustrate selected aspects related to using the medical device for treating esophageal atresia;

FIGS. 17-19 illustrate selected aspects of a medical device for treating esophageal atresia; and

FIG. 20 illustrates selected aspects of an alternative configuration of the medical device of FIGS. 17-19.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently - such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Some features and/or reference numbers may not be shown in each figure for clarity. Similarly, each and every element and/or reference number may not be discussed in detail with respect to each figure. Some reference numbers and/or features may be shown and/or described in other figures in more detail, and those reference numbers and/or features may be shown and/or identified in some figures merely for reference.

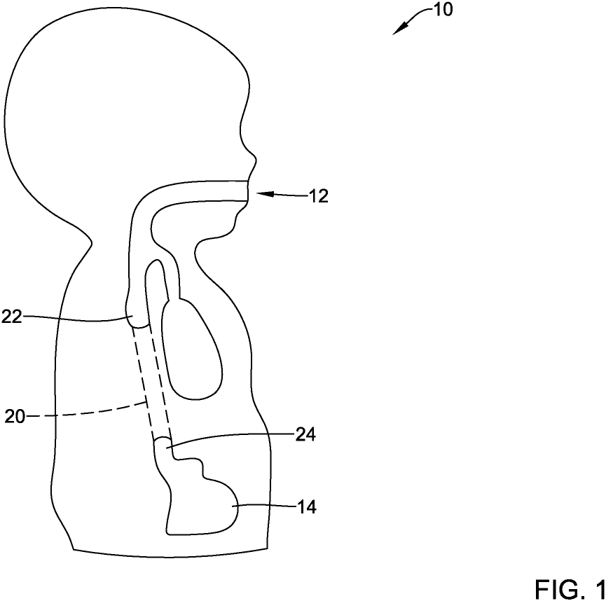

FIG. 1 illustrates aspects of an example of esophageal atresia, which is a congenital defect affecting a number of babies each year. As shown in FIG. 1, a patient 10 may be born with an esophagus 20 that is incomplete, resulting in the patient 10 having an upper esophageal pouch 22 connected to the mouth 12 and a lower esophageal pouch 24 connected to the stomach 14. The upper esophageal pouch 22 and the lower esophageal pouch 24 may be spaced apart from each other within the torso of the patient 10, such that the upper esophageal pouch 22 is unconnected to or discontinuous with the lower esophageal pouch 24. This disclosure describes medical devices and/or systems for treating pediatric esophageal atresia without subjecting the patient 10 to or with reduced long-term sedation and/or immobilization.

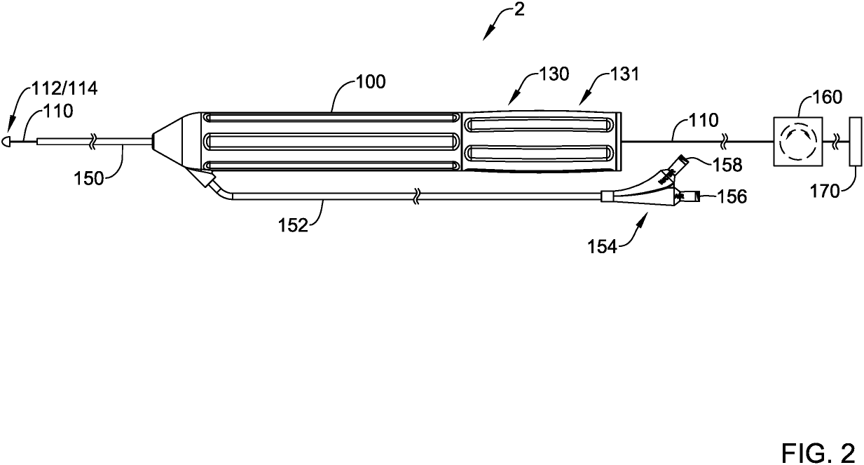

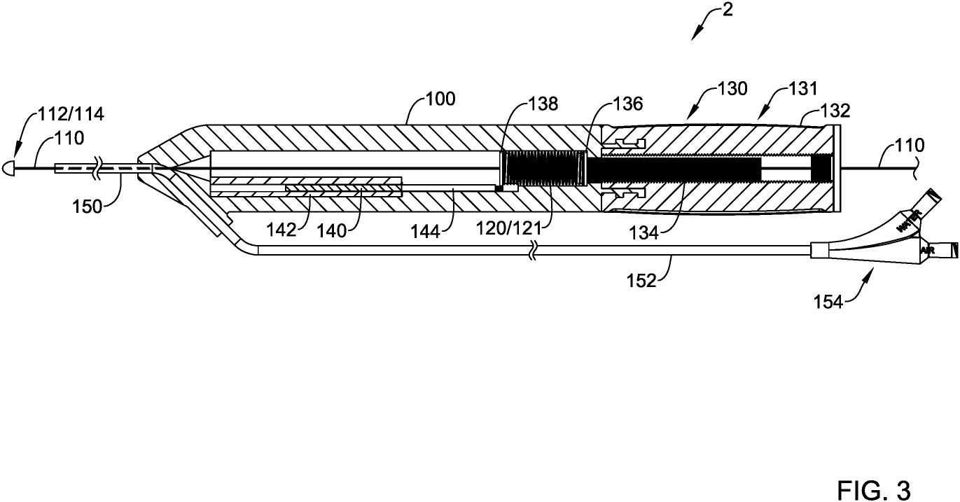

FIG. 2 is a side view and FIG. 3 is a partial cross-sectional view illustrating selected aspects of a medical device 2 for treating esophageal atresia. The medical device 2 may include a handle 100. In some embodiments, the handle 100 may be a generally elongate tubular structure having and/or capable of having elements disposed therein. Other configurations are also contemplated. Some suitable, but non-limiting, examples of materials for the handle 100, including but not limited to polymeric materials, metallic materials, and/or composite materials, are discussed below.

The medical device 2 may include an elongate shaft 110 extending distally from the handle 100. The elongate shaft 110 may be configured to extend through the mouth 12 of the patient 10 and into the upper esophageal pouch 22 of the patient 10. The elongate shaft 110 may include a distal end 112 configured to engage a distal end of the upper esophageal pouch 22. In at least some embodiments, the elongate shaft 110 and/or the distal end 112 of the elongate shaft 110 may include an atraumatic distal head 114 having a first outer diameter. In some embodiments, the first outer diameter of the atraumatic distal head 114 may be about 14 French (Fr) (about 4.67 millimeters (mm)), about 16 Fr (about 5.33 mm), about 18 Fr (about 6.0 mm), about 20 Fr (about 6.67 mm), or another suitable size depending on the size of the patient 10. In one example embodiment, the first outer diameter is about 20 Fr (about 6.67 mm). The first outer diameter and/or the atraumatic distal head 114 may be selected to best match the anatomical shape of the upper esophageal pouch 22.

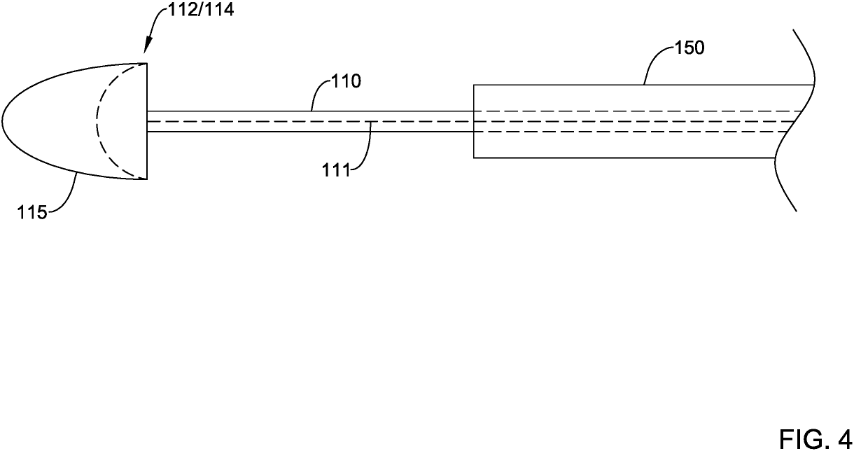

In some embodiments, the first outer diameter of the atraumatic distal head 114 may be variable. In some embodiments, the distal end 112 of the elongate shaft 110 and/or the atraumatic distal head 114 may include an expandable portion. In some alternative embodiments, the distal end 112 of the elongate shaft 110 and/or the atraumatic distal head 114 may include a balloon 115 and/or a balloon expandable portion at a distal tip, as seen in FIG. 4. The elongate shaft 110 may include an inflation lumen 111 extending therethrough. The balloon 115 and/or the balloon expandable portion at the distal tip is shown in FIG. 4 in an inflated configuration (e.g., pressurized to a preset maximum pressure), and in phantom in a partially deflated configuration (e.g., pressurized to a preset minimum pressure). In some embodiments, the balloon 115 and/or the balloon expandable portion at the distal tip may permit and/or provide an improved fit with and/or against the upper esophageal pouch 22. In some embodiments, the balloon 115 and/or the balloon expandable portion at the distal tip may be configured to shift to the deflated configuration (or to a completely deflated configuration) in response to movement of the patient and/or an increase in internal pressure within the balloon 115 and/or the balloon expandable portion at the distal tip above the preset maximum pressure. In some embodiments, the balloon 115 and/or the balloon expandable portion at the distal tip may be configured to expand longitudinally with no or minimal radial expansion when inflated from the partially deflated configuration to the inflated configuration. In some embodiments, an outer diameter of the balloon 115 and/or the balloon expandable portion at the distal tip may be variable. Other configurations are also contemplated.

Returning to FIGS. 2 and 3, the atraumatic distal head 114 may be configured to engage the distal end of the upper esophageal pouch 22 in a manner that substantially prevents cutting, tearing, abrasion, etc. of the tissue of the upper esophageal pouch 22. In some embodiments, the atraumatic distal head 114 may be generally mushroom-shaped, wherein the elongate shaft 110 forms a proximal stem of the atraumatic distal head 114, and a distal surface of the atraumatic distal head 114 may be generally curved and/or rounded from a distalmost extent of the atraumatic distal head 114 in a proximal direction. In some embodiments, the distal end 112 of the elongate shaft 110 and/or the atraumatic distal head 114 may include a pressure sensor and/or a force sensor disposed therein. In some embodiments, the pressure sensor and/or the force sensor may be configured to measure a force and/or pressure applied to the distal end of the upper esophageal pouch 22 by the distal end 112 and/or the atraumatic distal head 114.

The elongate shaft 110 may be generally flexible in a lateral direction to permit navigation through the mouth 12 of the patient 10 into the esophagus 20 and/or the upper esophageal pouch 22. The elongate shaft 110 may be generally rigid and/or stiff in an axial direction to promote pushability and/or transmission of force axially along the elongate shaft 110, as discussed herein. In some embodiments, the elongate shaft 110 may have a tubular elongate structure having at least one lumen extending therethrough. In some embodiments, the elongate shaft 110 may have a solid elongate structure. Other configurations, including combinations thereof, are also contemplated. Some suitable, but non-limiting, examples of materials for the elongate shaft 110, including but not limited to polymeric materials, metallic materials, and/or composite materials, are discussed below.

In some embodiments, the elongate shaft 110 may extend axially into and/or through the handle 100. In some embodiments, the elongate shaft 110 may extend proximal of the handle 100 and engage with a longitudinal advancement-retraction mechanism 160, such as a bidirectional longitudinal advancement and retraction mechanism (e.g., a reel, a coil, etc.), disposed proximal of the handle 100. In some embodiments, the longitudinal advancement-retraction mechanism 160 may include a rotating element disposed within a longitudinal advancement-retraction mechanism housing, wherein the elongate shaft 110 is configured to wind or coil around the rotating element as the elongate shaft 110 is translated axially relative to the longitudinal advancement-retraction mechanism 160 and/or the handle 100. In some embodiments, the longitudinal advancement-retraction mechanism 160 may include a spring mechanism or other self-biasing element disposed within the longitudinal advancement-retraction mechanism housing that is configured to bias the rotating element to “reel up” the elongate shaft 110 as the elongate shaft 110 is translated proximally relative to the longitudinal advancement-retraction mechanism 160 and/or the handle 100.

The pressure sensor and/or the force sensor may include a wire, a fiber optic wire, etc. extending within and/or through the elongate shaft 110, through the longitudinal advancement-retraction mechanism 160, to a monitoring device 170. In some embodiments, the monitoring device 170 may include a display indicating a value of the force and/or a pressure applied to esophageal tissue by the distal end 112 and/or the atraumatic distal head 114. In some embodiments, the monitoring device 170 may include and/or emit visual and/or audible alarms if the force exceeds a predetermined maximum force and/or if the force drops below a predetermined force threshold. In some embodiments, the monitoring device 170 may include and/or emit visual and/or audible alarms if the pressure exceeds a predetermined maximum pressure and/or if the pressure drops below a predetermined pressure threshold. In some embodiments, the monitoring device 170 may include and/or may be in electronic communication with a computer, a microprocessor, a network, a cloud computing service, or other communications equipment.

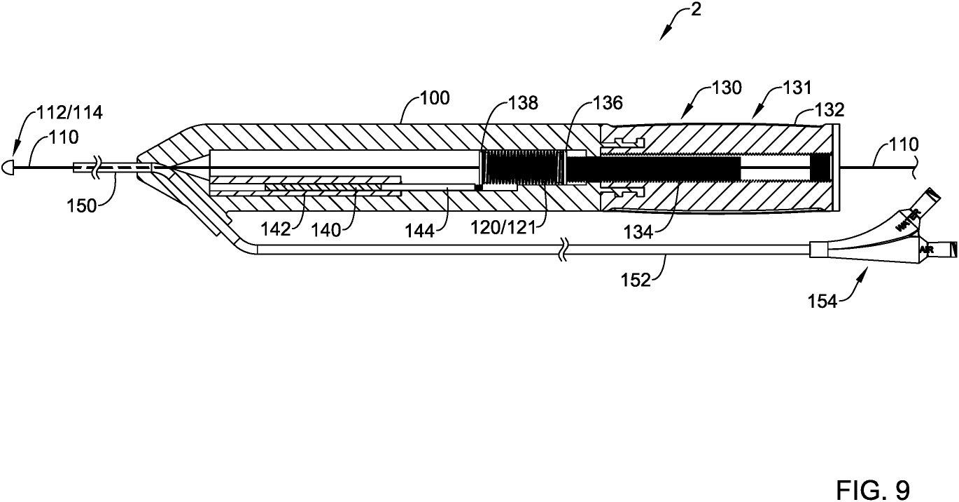

In some embodiments, the medical device 2 may include a force absorbing member 120 disposed within the handle 100, as seen in FIG. 3 for example. The force absorbing member 120 may be configured to permit the elongate shaft 110 to translate axially relative to the handle 100. In some embodiments, the force absorbing member 120 may be configured to permit the elongate shaft 110 to translate axially relative to the handle 100 in response to movements of the patient 10 (e.g., FIG. 9). In some embodiments, the force absorbing member 120 may be configured to exert a force along the elongate shaft 110 to apply the force to the distal end of the upper esophageal pouch 22 of the patient 10. In some embodiments, the pressure sensor and/or the force sensor disposed within the distal end 112 and/or the atraumatic distal head 114 may be configured to measure the force applied to the distal end of the upper esophageal pouch 22 by the force absorbing member 120. In some embodiments, the force absorbing member 120 may be configured to permit the elongate shaft 110 to translate axially relative to the handle 100 while maintaining the force against the distal end of the upper esophageal pouch 22. In some embodiments, the elongate shaft 110 may be fixedly secured relative to a distal end of the force absorbing member 120.

In some embodiments, the force may be applied constantly to the distal end of the upper esophageal pouch 22 of the patient 10. In some embodiments, the force may be varied over time. In some embodiments, the force may be applied cyclically to the distal end of the upper esophageal pouch 22 of the patient 10. For example, in some embodiments, the force may be applied to the distal end of the upper esophageal pouch 22 of the patient 10 for a first interval of time and the force may be removed for a second interval of time, wherein the first interval of time alternates with the second interval of time. In some embodiments, the force may be applied to the distal end of the upper esophageal pouch 22 of the patient 10 at an original value until a certain amount of growth of the upper esophageal pouch 22 occurs and/or the force drops below a certain amount from the original value, and then the force may be increased and/or re-applied back to the original value. In some embodiments, the force may be applied to the distal end of the upper esophageal pouch 22 of the patient 10 such that the force follows a cyclical wave (e.g., a sine wave, etc.), wherein the force is slowly increased over time and then slow decreased over time in a repeating fashion and/or pattern. Other configurations are also contemplated. In some embodiments, the cyclical application of force may allow and/or permit at least some tissue relaxation and/or reperfusion during the duration of process and/or procedure. In some embodiments, force may be applied randomly to the distal end of the upper esophageal pouch 22 of the patient 10 as the patient 10 moves. In some embodiments, the force may vary randomly about a target force value (e.g., the original force). In some embodiments, the force may be applied randomly to the distal end of the upper esophageal pouch 22 of the patient 10 about the target force value with a preset and/or user set maximum force value. Other configurations, including various combinations thereof, are also contemplated. In some embodiments, the force may be no more than 1.0 pounds of force. In some embodiments, the force may be no more than 0.8 pounds of force. In some embodiments, the force may be no more than 0.6 pounds of force. In some embodiments, the force may be no more than 0.5 pounds of force. Other configurations are also contemplated.

In some embodiments, the force absorbing member 120 may include and/or may be a spring 121. In some embodiments, the force absorbing member 120 may include and/or may be a compression spring. In some embodiments, the force absorbing member 120 may include and/or may be a torsion spring. In some embodiments, the force absorbing member 120 may include and/or may be a spring held in tension. In some embodiments, the force absorbing member 120 may include and/or may be a fluid or a gas. In some embodiments, the force absorbing member 120 may include and/or may be an elastic member. In some embodiments, the force absorbing member 120 and/or the spring 121 may be compressible and/or may be resiliently compressible. In some embodiments, the force absorbing member 120 and/or the spring 121 may be axially compressible and/or may be axially resilient. Other configurations are also contemplated.

In some embodiments, the force absorbing member 120 and/or the spring 121 may be movably disposed within the handle 100. In some embodiments, the force absorbing member 120 and/or the spring 121 may be translatable within the handle 100. In some embodiments, the force absorbing member 120 and/or the spring 121 may include a longitudinal axis oriented substantially parallel to a longitudinal axis of the handle 100 and/or the elongate shaft 110. In some embodiments, the force absorbing member 120 and/or the spring 121 may be axially translatable within the handle 100. In some embodiments, the force absorbing member 120 and/or the spring 121 may be rotatable within the handle 100. In some embodiments, the force absorbing member 120 and/or the spring 121 may be translatable in a transverse direction with respect to the handle 100. Other configurations, including combinations thereof, are also contemplated. Some suitable, but non-limiting, examples of materials for the force absorbing member 120 and/or the spring 121, including but not limited to polymeric and/or metallic materials, are discussed below.

The medical device 2 and/or the handle 100 may include an adjustment mechanism 130 configured to adjust the force applied to the distal end of the upper esophageal pouch 22 by the elongate shaft 110, and corresponding distal end 112 and/or atraumatic distal head 114 via force absorbing member 120 and/or the spring 121. In some embodiments, the adjustment mechanism 130 may be coupled to the handle 100. In at least some embodiments, the adjustment mechanism 130 may be configured to translate the force absorbing member 120 and/or the spring 121 within the handle 100. In some embodiments, the adjustment mechanism 130 may be configured to axially translate and/or compress the force absorbing member 120 and/or the spring 121 within the handle 100.

In some embodiments, the medical device 2 and/or the handle 100 may include an axial translation mechanism 131 configured to adjust the force applied to the distal end of the upper esophageal pouch 22 by the force absorbing member 120 and/or the spring 121. In some embodiments, the adjustment mechanism 130 may be the axial translation mechanism 131. In some embodiments, the adjustment mechanism 130 and/or the axial translation mechanism 131 may include a knob 132. The knob 132, in some instances, may be disposed proximate a proximal end of the handle 100. In some embodiments, the knob 132 may be disposed at the proximal end of the handle 100. In some embodiments, the knob 132 may extend proximal of the handle 100. Other positions are also contemplated. In some embodiments, the adjustment mechanism 130, the axial translation mechanism 131, and/or the knob 132 may be rotatable with respect to the handle 100. In some embodiments, the adjustment mechanism 130, the axial translation mechanism 131, and/or the knob 132 may be fixed axially with respect to the handle 100. In some embodiments, the adjustment mechanism 130, the axial translation mechanism 131, and/or the knob 132 is prevented from axial movement relative to the handle 100. In some embodiments, the adjustment mechanism 130, the axial translation mechanism 131, and/or the knob 132 may be configured to translate axially with respect to the handle 100 and may be non-rotatable with respect to the handle 100. Other configurations are also contemplated.

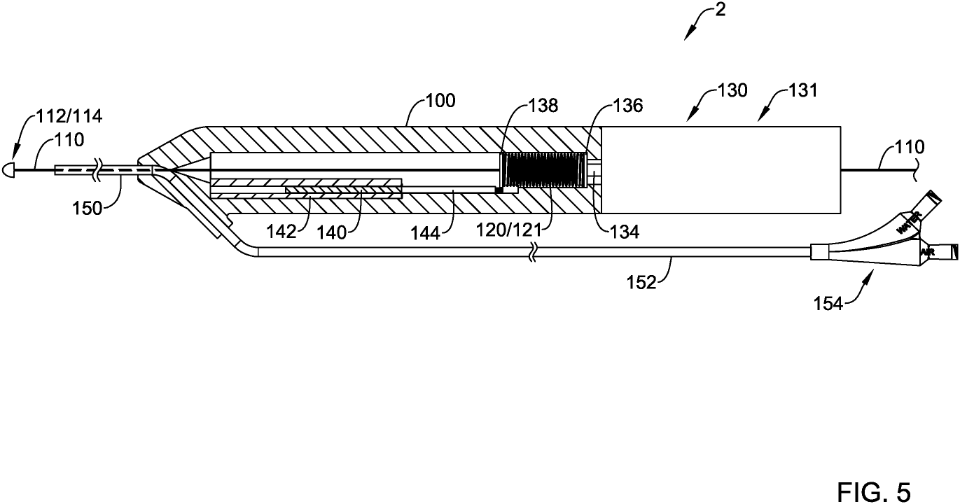

For simplicity and clarity, the figures are illustrated with the knob 132 as the adjustment mechanism 130 and/or the axial translation mechanism 131. However, other configurations are also contemplated, as shown and described with respect to FIG. 5.

FIG. 5 schematically illustrates the adjustment mechanism 130 and/or the axial translation mechanism 131 coupled to the handle 100. In some embodiments, the adjustment mechanism 130 and/or the axial translation mechanism 131 may include a fluid and/or be adjusted or actuated by a fluid (e.g., water, saline, a biocompatible fluid, etc.). In some embodiments, the adjustment mechanism 130 and/or the axial translation mechanism 131 may include a gas and/or be adjusted or actuated by a gas (e.g., air, a biocompatible gas, etc.). In some embodiments, the adjustment mechanism 130 and/or the axial translation mechanism 131 may include a screw thread and/or be adjusted or actuated by a screw thread. In some embodiments, the adjustment mechanism 130 and/or the axial translation mechanism 131 may include a motorized adjustment and/or be adjusted or actuated by a motorized adjustment. Other configurations, including combinations thereof, are also contemplated. In some embodiments, the adjustment mechanism 130 and/or the axial translation mechanism 131 may be substantially incompressible in an axial direction.

In some embodiments, the adjustment mechanism 130 and/or the axial translation mechanism 131 may include a shaft 134. In at least some embodiments, the shaft 134 may include and/or may be a threaded shaft. In some embodiments, the shaft 134 may extend distally from the knob 132 within the handle 100. In some embodiments, a distal portion of the shaft 134 and/or the threaded shaft may include a first flange 136 abutting a proximal end of the force absorbing member 120 and/or the spring 121. In some embodiments, the force absorbing member 120 and/or the spring 121 may be fixedly attached to the first flange 136. In some embodiments, actuation of the adjustment mechanism 130 and/or the axial translation mechanism 131 may translate the first flange 136 within the handle 100 proximally or distally. In some embodiments, actuation of the adjustment mechanism 130 and/or the axial translation mechanism 131 may axially translate the first flange 136 within the handle 100 proximally or distally. In some embodiments, rotation of the knob 132 may translate the first flange 136 within the handle 100 proximally or distally. In some embodiments, rotation of the knob 132 may axially translate the first flange 136 within the handle 100 proximally or distally. Some suitable, but non-limiting, examples of materials for the knob 132, including but not limited to polymeric and/or metallic materials, are discussed below.

In at least some embodiments, the elongate shaft 110 may extend axially through the knob 132 and/or may be movable with respect to the knob 132. In at least some embodiments, the elongate shaft 110 may extend axially through the shaft 134 and/or the first flange 136. In some embodiments, the elongate shaft 110 may extend through the force absorbing member 120 and/or the spring 121. In some embodiments, the elongate shaft 110 may extend axially through the force absorbing member 120 and/or the spring 121.

In some embodiments, the medical device 2 may include a second flange 138 abutting the distal end of the force absorbing member 120 and/or the spring 121. In some embodiments, the second flange 138 may be fixedly attached to the force absorbing member 120 and/or the spring 121. In some embodiments, the elongate shaft 110 may extend through the second flange 138. In some embodiments, the elongate shaft 110 may extend axially through the second flange 138. In some embodiments, the elongate shaft 110 may be axially fixed relative to the distal end of the force absorbing member 120 and/or the spring 121. In some embodiments, the elongate shaft 110 may be fixedly attached to the second flange 138.

The knob 132 may include threading that threadably engages or mates with the threading on the shaft 134 such that rotation of the knob 132 (which is not permitted to longitudinally move relative to the handle 100) causes longitudinal movement of the shaft 134. For example, rotation of the knob 132 is a first rotational direction (e.g., clockwise) causes or results in longitudinal movement of the shaft 134 in a distal direction relative to the handle 100, and rotation of the knob 132 in a second rotation direction (e.g., counterclockwise) opposite the first rotational direction causes or results in longitudinal movement of the shaft 134 in a proximal direction relative to the handle 100. Accordingly, rotation of the knob 132 in the first rotational direction moves the first flange 136 distally within the handle 100. Movement of the first flange 136 distally may move the first flange 136 toward the second flange 138, thereby compressing the spring 121 when the distal end 112 and/or atraumatic distal head 114 is pressed against the distal end of the upper esophageal pouch 22, and thus increasing the force exerted on the distal end of the upper esophageal pouch 22 by the spring 121.

During use, as the upper esophageal pouch 22 grows or stretches, the second flange 138 may move distally relative to the first flange 136, as the spring 121 expands longitudinally. The spring 121 may slowly axially expand (e.g., over a course of several days) as the upper esophageal pouch 22 stretches while the distal end 112 and/or atraumatic distal head 114 remains engaged with the distal end of the upper esophageal pouch 22. Movement of the spring (e.g., axial compression and axial expansion) between the first flange 136 and the second flange 138 may generate cyclic movement of the elongate shaft (and the distal end 112 and/or atraumatic distal head 114 associated therewith) relative to the handle 100 while maintaining force and pressure against the distal end of the upper esophageal pouch 22. It is noted that in some instances, the force and/or pressure applied to the distal end of the upper esophageal pouch 22 may slowly decrease over time as the spring 121 slowly axially expands.

In some embodiments, the medical device 2 may include a position sensor 140 disposed within the handle 100 or at a different location of the medical device 2. In some embodiments, the position sensor 140 may be configured to detect an axial position of the elongate shaft 110 relative to the handle 100. In some embodiments, the position sensor 140 may include and/or may be disposed within a position sensor housing 142. The position sensor housing 142 may be secured to and/or within the handle 100. In some embodiments, the position sensor housing 142 may be removable from the handle 100. In some embodiments, the position sensor housing 142 may be secured in a fixed position within the handle 100 and/or the position sensor housing 142 may be unable to move within the handle 100.

In some embodiments, the position sensor 140 may be movable within and/or with respect to the position sensor housing 142. In some embodiments, the position sensor 140 may be axially movable within and/or with respect to the position sensor housing 142. Other configurations are also contemplated. In some embodiments, the medical device 2 and/or the position sensor 140 may include a position sensor shaft 144 fixedly attached to the position sensor 140. In some embodiments, the position sensor shaft 144 may be fixedly secured relative to the distal end of the force absorbing member 120 and/or the spring 121. In some embodiments, the position sensor shaft 144 may be secured to the second flange 138. In some embodiments, the position sensor shaft 144 may include a distal end fixedly attached to the position sensor 140 and a proximal end fixedly attached to the second flange 138. Other configurations are also contemplated. Movement and/or translation of the position sensor 140 relative to the position sensor housing 142 and/or handle 100 may correspond to longitudinal displacement of the distal end 112 and/or the atraumatic distal head 114 relative to the handle 100 and/or a distal end of an elongate tubular member 150 through which the elongate shaft 110 travels through. As such, translation of the force absorbing member 120 and/or the spring 121 and/or the second flange 138 within and/or relative to the handle 100 will result in corresponding movement and/or translation of the position sensor 140, thereby enabling monitoring of axial translation of the elongate shaft 110, and thus elongation of the upper esophageal pouch 22, over time.

In some embodiments, the medical device 2 may include an elongate tubular member 150 extending distally from the handle 100. In at least some embodiments, the elongate tubular member 150 may be configured to extend through the mouth 12 of the patient 10 and into the upper esophageal pouch 22 of the patient 10. At least a portion of the elongate shaft 110 may be movably disposed within at least a portion of the elongate tubular member 150. The elongate tubular member 150 may have a second outer diameter less than the first outer diameter of the atraumatic distal head 114. In some embodiments, the second outer diameter may be about 6 French (Fr) (about 2.0 millimeters (mm)), about 8 Fr (about 2.67 mm), about 10 Fr (about 3.33 mm), or about 12 Fr (4.0 mm). In some embodiments, the medical device 2 may permit separate support of respiration by other devices alongside and/or while the medical device 2 is in place without impeding respiratory treatment. For example, in some embodiments, the elongate tubular member 150 may be sized to permit intubation alongside the elongate tubular member 150. In some alternative embodiments, the second outer diameter of the elongate tubular member 150 may be of sufficient size to accommodate a respiration tube disposed within the elongate tubular member 150.

In some embodiments, the medical device 2 may include a second elongate tubular member 152 extending proximally from the handle 100. In some embodiments, the elongate tubular member 150 and the second elongate tubular member 152 may be formed as a single and/or monolithic tubular structure. As such, in some embodiments, the second elongate tubular member 152 may be a proximal portion of the elongate tubular member 150 rather than a separate element or structure. Other configurations are also contemplated. Some suitable, but non-limiting, examples of materials for the elongate tubular member 150 and/or the second elongate tubular member 152, including but not limited to polymeric and/or metallic materials, are discussed below.

The elongate tubular member 150 and the second elongate tubular member 152 may include and/or define a continuous lumen extending therethrough. In embodiments where the elongate tubular member 150 is separately formed from the second elongate tubular member 152, a lumen of the elongate tubular member 150 may be in fluid communication with a lumen of the second elongate tubular member 152. In some embodiments, the handle 100 may include a lumen in fluid communication with the lumen of the elongate tubular member 150 and the lumen of the second elongate tubular member 152 to form the continuous lumen.

In some embodiments, the medical device 2 may include a Y-adapter 154 at and/or fixedly attached to a proximal end of the elongate tubular member 150 and/or the second elongate tubular member 152. The Y-adapter 154 may be in fluid communication with the lumen of the second elongate tubular member 152 and/or the continuous lumen extending through the elongate tubular member 150 and the second elongate tubular member 152. In some embodiments, the Y-adapter 154 may include a first port 156 and a second port 158. The first port 156 may be in fluid communication with the lumen of the second elongate tubular member 152 and/or the continuous lumen extending through the elongate tubular member 150 and the second elongate tubular member 152. The second port 158 may be in fluid communication with the lumen of the second elongate tubular member 152 and/or the continuous lumen extending through the elongate tubular member 150 and the second elongate tubular member 152. Some suitable, but non-limiting, examples of materials for the Y-adapter 154, the first port 156, and/or the second port 158, including but not limited to polymeric and/or metallic materials, are discussed below.

In some embodiments, the first port 156 may be configured to connect to a source of suction and/or a vacuum source (e.g., air, etc.). In at least some embodiments, the first port 156 may be used in cooperation with the lumen of the second elongate tubular member 152 and/or the continuous lumen extending through the elongate tubular member 150 and the second elongate tubular member 152 to suction fluid, mucous, debris, etc. from the distal end of the upper esophageal pouch 22. In some embodiments, the medical device 2 may be configured to treat esophageal atresia and for suction of mucous and/or saliva from the distal end of the upper esophageal pouch 22 simultaneously, thereby preventing mucous and/or saliva from entering the trachea, which could lead to potential disruption of adequate respiration. As such, the medical device 2 is able to help sustain the life of the patient while treating the esophageal atresia.

In some embodiments, the second port 158 may be configured to connect to a source of irrigation (e.g., water, saline, etc.). In some embodiments, the second port 158 may be used in cooperation with the lumen of the second elongate tubular member 152 and/or the continuous lumen extending through the elongate tubular member 150 and the second elongate tubular member 152 to irrigate the distal end of the upper esophageal pouch 22. In at least some embodiments, the second port 158 may be used to introduce a separate irrigation tube into the lumen of the second elongate tubular member 152 and/or the continuous lumen extending through the elongate tubular member 150 and the second elongate tubular member 152 to irrigate the distal end of the upper esophageal pouch 22, thereby permitting the lumen of the second elongate tubular member 152 and/or the continuous lumen extending through the elongate tubular member 150 and the second elongate tubular member 152, in cooperation with the first port 156, to be simultaneously used for suction of mucous and/or saliva from the distal end of the upper esophageal pouch 22. Other configurations are also contemplated.

In some embodiments, irrigation of the distal end of the upper esophageal pouch 22 may be periodic and/or performed as needed. In some embodiments, suction of mucous and/or saliva from the distal end of the upper esophageal pouch 22 may be continuous. Other configurations are also contemplated.

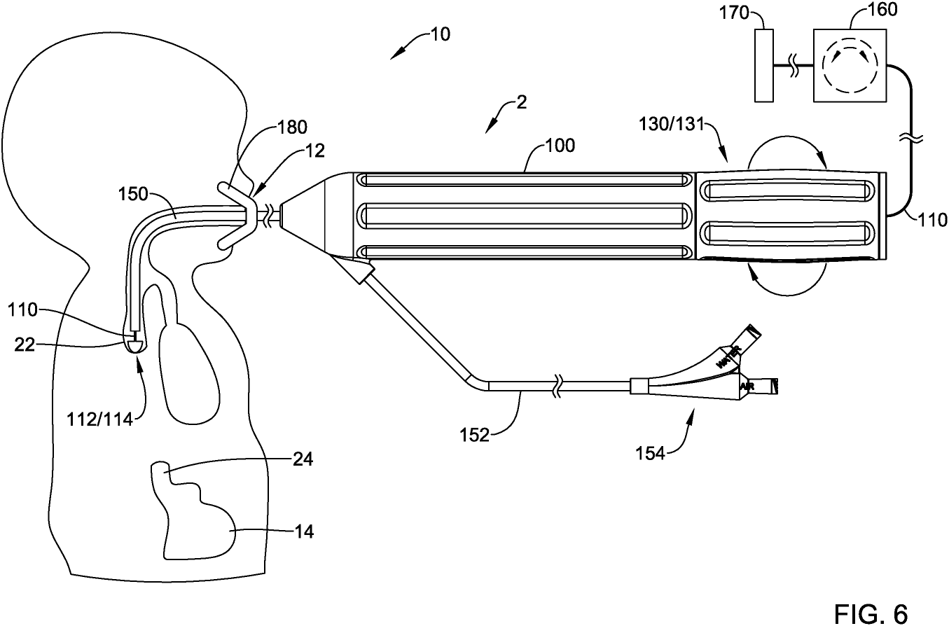

A method of treating esophageal atresia may include inserting the medical device 2, as described herein, into the esophagus 20 through the mouth 12 of the patient 10, as shown in FIG. 6. The method may include positioning the distal end 112 of the elongate shaft 110 and/or the atraumatic distal head 114 at and/or against the distal end (e.g., the closed end) of the upper esophageal pouch 22.

The method may include fixing the medical device 2 and/or the elongate tubular member 150 in place relative to the patient 10, such as relative to the mouth 12, and/or the distal end of the upper esophageal pouch 22 with the distal end 112 and/or atraumatic distal head 114 at and/or against the distal closed end of the upper esophageal pouch 22. In some embodiments, the medical device 2 and/or the elongate tubular member 150 may be fixed in place using a fixation element 180. In some embodiments, the fixation element 180 may be secured to the face of the patient 10, such as with adhesive tape. In some embodiments, the fixation element 180 may include a headgear or a mouthpiece secured to the head of the patient 10. In some embodiments, the fixation element 180 may include a mask or other structure configured to at least partially surround and/or encompass the head of the patient 10. In some embodiments, the medical device 2 and/or the elongate tubular member 150 may be secured to the patient 10 and/or may be secured relative to the patient 10, the mouth 12, and/or the distal end of the upper esophageal pouch 22 by securing the medical device 2 and/or the elongate tubular member 150 to another medical device or product such as a NeoBar® from Neotech Products. Other suitable medical devices and/or products are also contemplated.

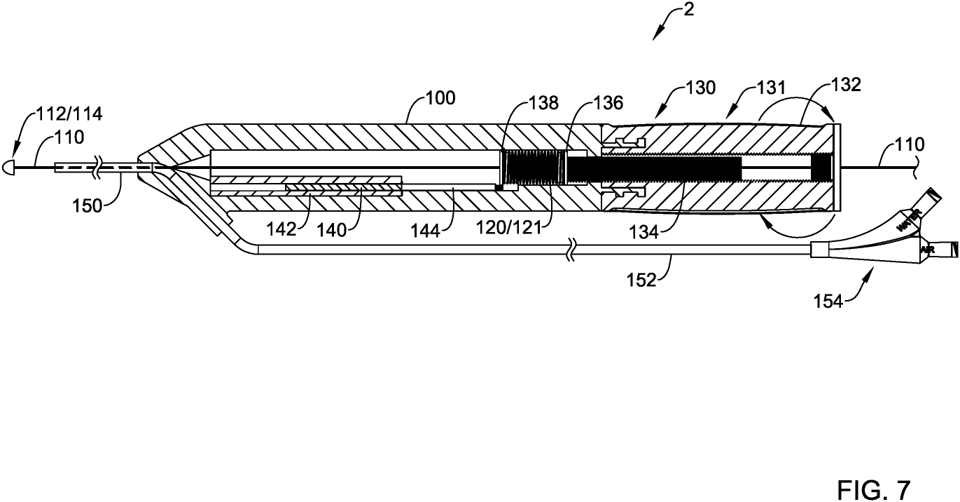

In some embodiments, the method may include actuating the adjustment mechanism 130 and/or the axial translation mechanism 131 to adjust the force applied to the distal end of the upper esophageal pouch 22 by the force absorbing member 120 and/or the spring 121. As seen in FIGS. 6 and 7, actuation of the adjustment mechanism 130 and/or the axial translation mechanism 131, and/or rotation of the knob 132 relative to the handle 100, may translate the first flange 136 within and/or relative to the handle 100 distally to compress the force absorbing member 120 and/or the spring 121, thereby increasing the force exerted against the distal end of the upper esophageal pouch 22 by the force absorbing member 120 and/or the spring 121. Since the distal end 112 of the elongate shaft 110 and/or the atraumatic distal head 114 is positioned against the distal end of the upper esophageal pouch 22, the second flange 138 is prevented from translating within and/or relative to the handle 100.

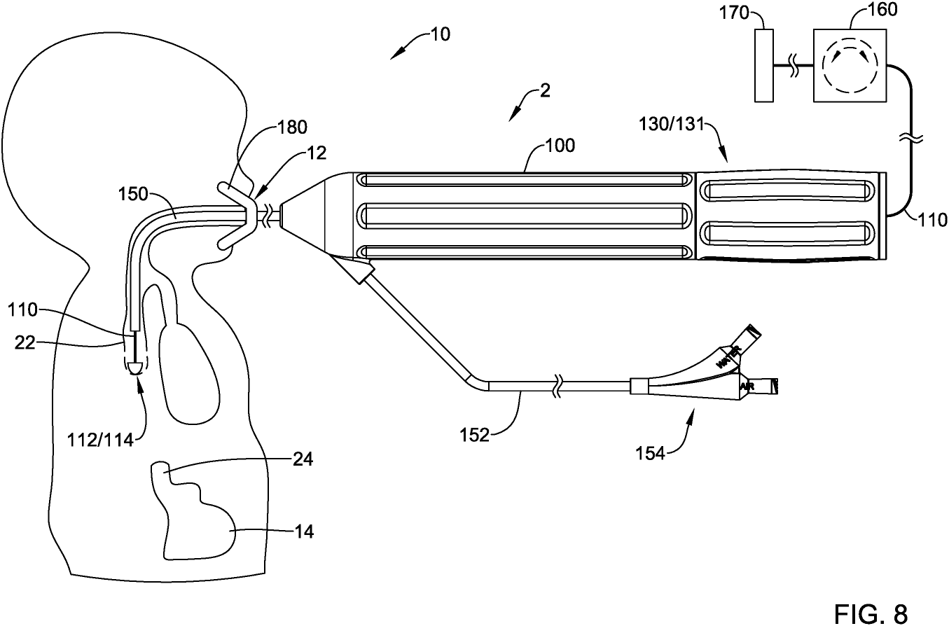

Over time, the distal end of the upper esophageal pouch 22 will grow and/or stretch as the distal end of the upper esophageal pouch 22 is subjected to the force from the force absorbing member 120 and/or the spring 121, as shown in FIG. 8. As described herein, the distal end 112 of the elongate shaft 110 and/or the atraumatic distal head 114 may include a pressure sensor and/or a force sensor disposed therein. The pressure sensor and/or the force sensor may include a wire, a fiber optic wire, etc. extending within and/or through the elongate shaft 110, through the longitudinal advancement-retraction mechanism 160, to a monitoring device 170. In some embodiments, the monitoring device 170 may include a display indicating a value of the force and/or pressure applied. In some embodiments, the monitoring device 170 may include and/or emit visual and/or audible alarms if the force exceeds a predetermined maximum force and/or a user set maximum force. In some embodiments, the monitoring device 170 may include and/or emit visual and/or audible alarms if the force drops below a predetermined force threshold and/or a user set force threshold. In some embodiments, the monitoring device 170 may include and/or emit visual and/or audible alarms if the pressure exceeds a predetermined maximum pressure and/or a user set maximum pressure. In some embodiments, the monitoring device 170 may include and/or emit visual and/or audible alarms if the pressure drops below a predetermined pressure threshold and/or a user set pressure threshold. Other configurations are also contemplated.

As the distal end of the upper esophageal pouch 22 grows and/or stretches, the second flange 138 is biased away from the first flange 136 by the force absorbing member 120 and/or the spring 121, which slowly expands, decompresses, and/or resiliently expands, as seen in FIG. 9. As the second flange 138 translates within and/or relative to the handle 100 distally, the position sensor shaft 144 is translated within and/or relative to the handle 100 and simultaneously and/or consequently translates the position sensor 140 within and/or relative to the position sensor housing 142 and/or the handle 100. Translation of the force absorbing member 120 and/or the spring 121 and/or the second flange 138 within and/or relative to the handle 100 will result in corresponding movement and/or translation of the position sensor 140, thereby enabling continuous and/or consistent monitoring of axial translation of the elongate shaft 110, and thus elongation of the upper esophageal pouch 22, over time. In some embodiments, a change in voltage of the position sensor 140 and/or within the position sensor housing 142 may be used to determine the location and/or the position of the position sensor 140 (and by extension the position of the elongate shaft 110, the distal end 112 of the elongate shaft 110, and/or the atraumatic distal head 114) relative to the handle 100. In some embodiments, a visual indicator or mechanism (e.g., outside of the handle 100) may be used to determine the location and/or the position of the elongate shaft 110, the distal end 112 of the elongate shaft 110, and/or the atraumatic distal head 114 relative to the handle 100. Such position, and thus the change in position of the elongate shaft 110, the distal end 112 of the elongate shaft 110, and/or the atraumatic distal head 114 relative to the handle 100 over time, may be used to determine the amount the upper esophageal pouch 22 has stretched over a period of time and/or the rate of elongation of the upper esophageal pouch 22.

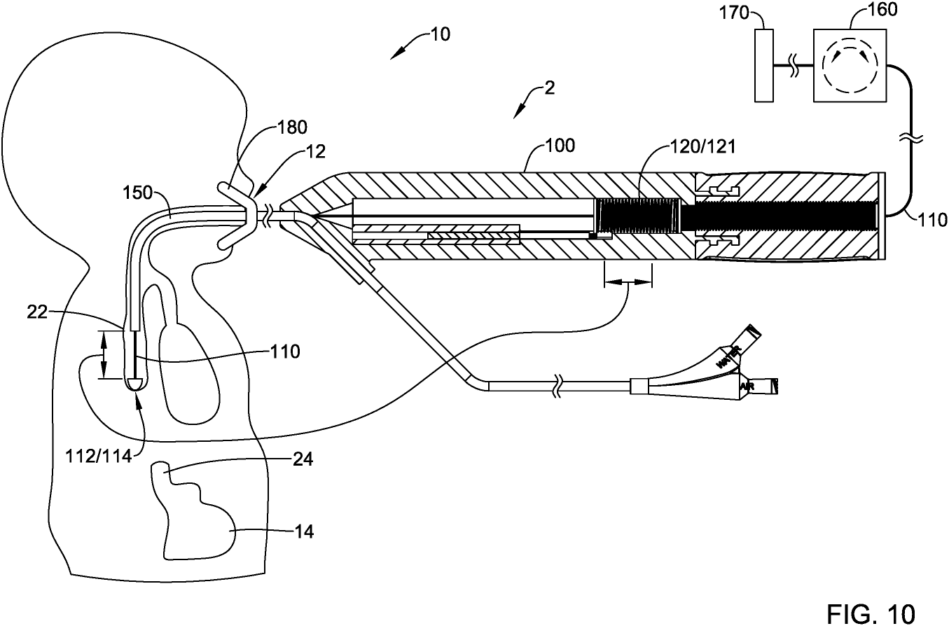

As shown via arrows in FIG. 10, the force absorbing member 120 and/or the spring 121 may be configured to permit the elongate shaft 110 to translate axially relative to the handle 100. In some embodiments, the force absorbing member 120 and/or the spring 121 may be configured to permit the elongate shaft 110 to translate axially relative to the handle 100 in response to movements of the patient 10. Accordingly, as the patient 10 moves due to respiration, coughing, sneezing, crying, etc. the force absorbing member 120 and/or the spring 121 may permit corresponding movement of the elongate shaft 110 in an oscillatory manner (e.g., back and forth in an axial direction), thereby reducing the potential and/or opportunity for rupture for the upper esophageal pouch 22. In some embodiments, permitting axial oscillatory movement of the elongate shaft 110 relative to the handle, and thus the distal end of the upper esophageal pouch 22, may also create and/or permit some occasional variation in the force applied to the distal end of the upper esophageal pouch 22 by the force absorbing member 120 and/or the spring 121. Thus, the elongate shaft 110 may cyclically move longitudinally or axially relative to the elongate tubular member 150 and/or handle 100 by cyclically absorbing some longitudinal movement by the force absorbing member 120 and/or the spring 121. Such variation may permit and/or encourage reperfusion of the tissue at the distal end of the upper esophageal pouch 22, thereby reducing tissue morbidity and/or necrosis while providing sufficient force to induce growth and/or stretch of the distal end of the upper esophageal pouch 22 over time.

As the patient 10 moves and/or the elongate shaft 110 translates axially relative to the handle 100, the elongate shaft 110 may be taken up by and/or coiled by the longitudinal advancement-retraction mechanism 160 and/or released from and/or uncoiled from the longitudinal advancement-retraction mechanism 160, thereby preventing kinking of, damage to, etc. the elongate shaft 110 and/or the wire, the fiber optic wire, etc. disposed therein. The longitudinal advancement-retraction mechanism 160 may also permit less of the elongate shaft 110 to be hanging freely outside of the handle 100, where the elongate shaft 110 may become tangled with other devices and/or equipment, etc.

In some embodiments, the force absorbing member 120 and/or the spring 121 may be configured to exert a force along the elongate shaft 110 to apply the force to the distal end of the upper esophageal pouch 22 of the patient 10. In some embodiments, the pressure sensor and/or the force sensor disposed within the distal end 112 and/or the atraumatic distal head 114 may be configured to measure the force and/or pressure applied to the distal end of the upper esophageal pouch 22 by the force absorbing member 120 and/or the spring 121. In some embodiments, the force absorbing member 120 and/or the spring 121 may be configured to permit the elongate shaft 110 to translate axially relative to the handle 100 while maintaining the force against the distal end of the upper esophageal pouch 22.

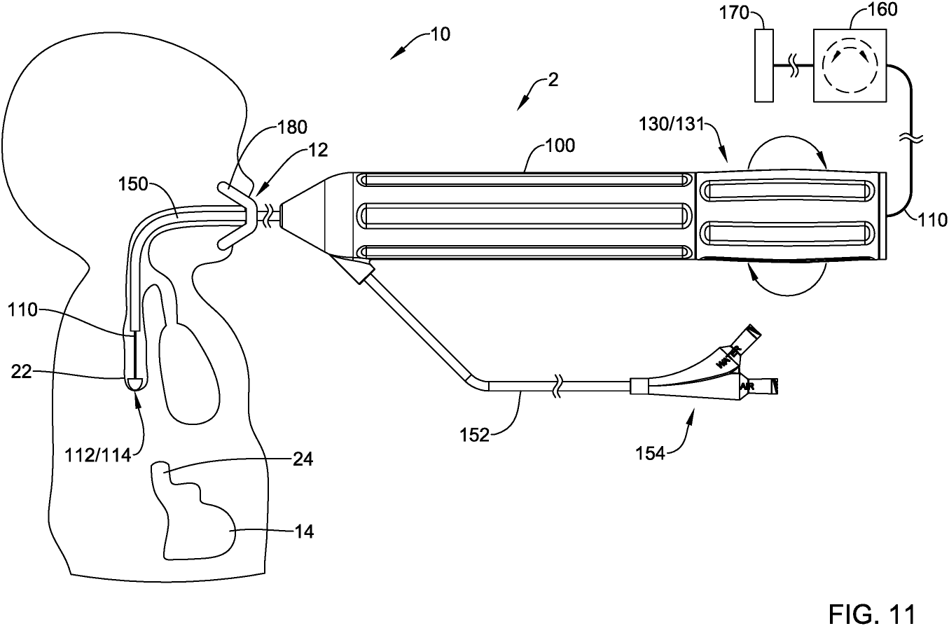

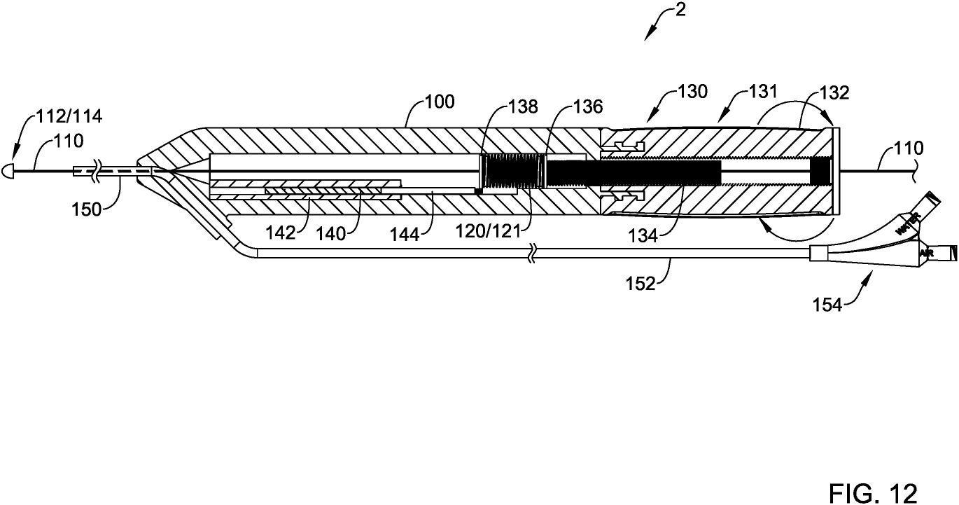

In some embodiments, the method may include monitoring the force applied to the distal end of the upper esophageal pouch 22 over time. In some embodiments, the method may include monitoring axial translation of the elongate shaft 110 over time via the position sensor 140. In some embodiments, the method may include actuating the adjustment mechanism 130 and/or the axial translation mechanism 131, and/or rotating the knob 132, as seen in FIGS. 11 and 12, to increase the force applied to the distal end of the upper esophageal pouch 22 when the force drops below the predetermined force threshold or when axial translation of the elongate shaft 110 reaches a predetermined translation threshold.

In some embodiments, the predetermined force threshold may be 0.5 pounds of force. In some embodiments, the predetermined force threshold may be 0.4 pounds of force. In some embodiments, the predetermined force threshold may be 0.3 pounds of force. In some embodiments, the predetermined force threshold may be 0.2 pounds of force. In some embodiments, the predetermined force threshold may be 0.1 pounds of force. Other configurations and/or values are also contemplated.

In some embodiments, the predetermined translation threshold may be 1.0 centimeters (cm). In some embodiments, the predetermined translation threshold may be 1.5 cm. In some embodiments, the predetermined translation threshold may be 2.0 cm. In some embodiments, the predetermined translation threshold may be 2.5 cm. In some embodiments, the predetermined translation threshold may be 3.0 cm. In some embodiments, the predetermined translation threshold may be 3.5 cm. In some embodiments, the predetermined translation threshold may be 4.0 cm. In some embodiments, the predetermined translation threshold may be 4.5 cm. In some embodiments, the predetermined translation threshold may be 5.0 cm. Other configurations and/or values are also contemplated.

As seen in FIGS. 11 and 12, actuation of the adjustment mechanism 130 and/or the axial translation mechanism 131, and/or rotation of the knob 132 relative to the handle 100, may translate the first flange 136 within and/or relative to the handle 100 to compress the force absorbing member 120 and/or the spring 121, thereby increasing the force exerted against the distal end of the upper esophageal pouch 22 by the force absorbing member 120 and/or the spring 121. Since the distal end 112 of the elongate shaft 110 and/or the atraumatic distal head 114 is positioned against the distal end of the upper esophageal pouch 22, the second flange 138 is prevented from translating within and/or relative to the handle 100.

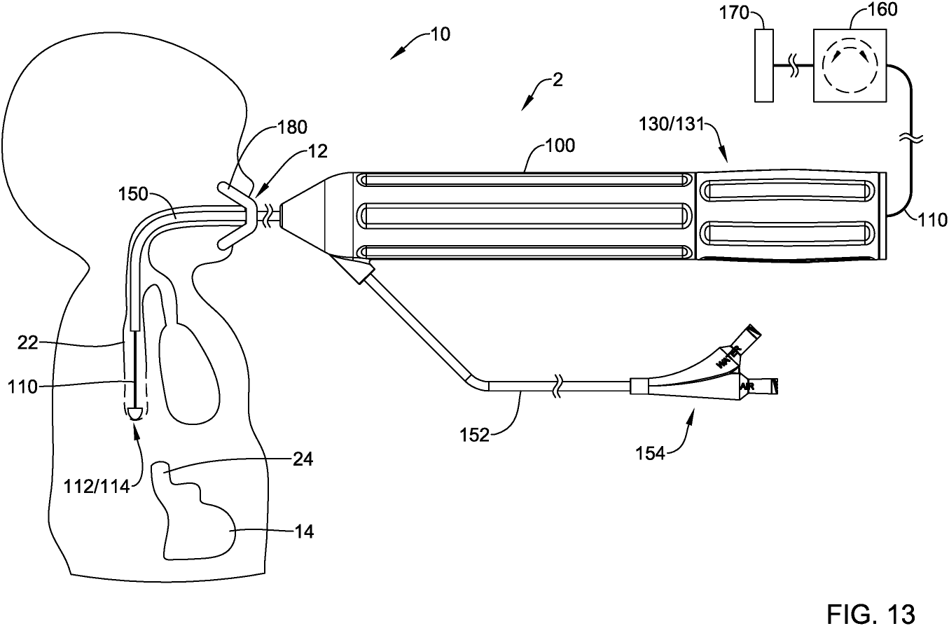

Over time, the distal end of the upper esophageal pouch 22 will grow and/or stretch as the distal end of the upper esophageal pouch 22 is subjected to the force from the force absorbing member 120 and/or the spring 121, as shown in FIG. 13. As described herein, the distal end 112 of the elongate shaft 110 and/or the atraumatic distal head 114 may include a pressure sensor and/or a force sensor disposed therein. The pressure sensor and/or the force sensor may include a wire, a fiber optic wire, etc. extending within and/or through the elongate shaft 110, through the longitudinal advancement-retraction mechanism 160, to a monitoring device 170. In some embodiments, the monitoring device 170 may include a display indicating a value of the force and/or pressure applied to the esophageal tissue. In some embodiments, the monitoring device 170 may include and/or emit visual and/or audible alarms if the force exceeds a predetermined maximum force and/or if the force drops below a predetermined force threshold. In some embodiments, the monitoring device 170 may include and/or emit visual and/or audible alarms if the pressure exceeds a predetermined maximum pressure and/or if the pressure drops below a predetermined pressure threshold. Other configurations are also contemplated.

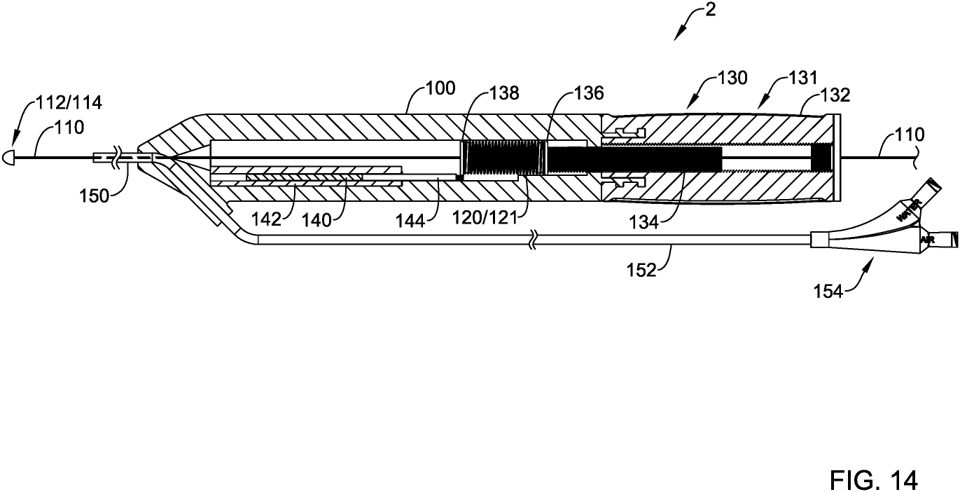

As the distal end of the upper esophageal pouch 22 grows and/or stretches, the second flange 138 is biased away from the first flange 136 by the force absorbing member 120 and/or the spring 121, which slowly expands, decompresses, and/or resiliently expands, as seen in FIG. 14. As the second flange 138 translates within and/or relative to the handle 100, the position sensor shaft 144 is translated within and/or relative to the handle 100 and simultaneously and/or consequently translates the position sensor 140 within and/or relative to the position sensor housing 142 and/or the handle 100. Translation of the force absorbing member 120 and/or the spring 121 and/or the second flange 138 within and/or relative to the handle 100 will result in corresponding movement and/or translation of the position sensor 140, thereby enabling continuous and/or consistent monitoring of axial translation of the elongate shaft 110, and thus elongation of the upper esophageal pouch 22 over time. The monitoring device 170 may calculate and/or display the amount of growth or elongation the upper esophageal pouch 22 has attained and/or the rate of growth or elongation of the upper esophageal pouch 22.

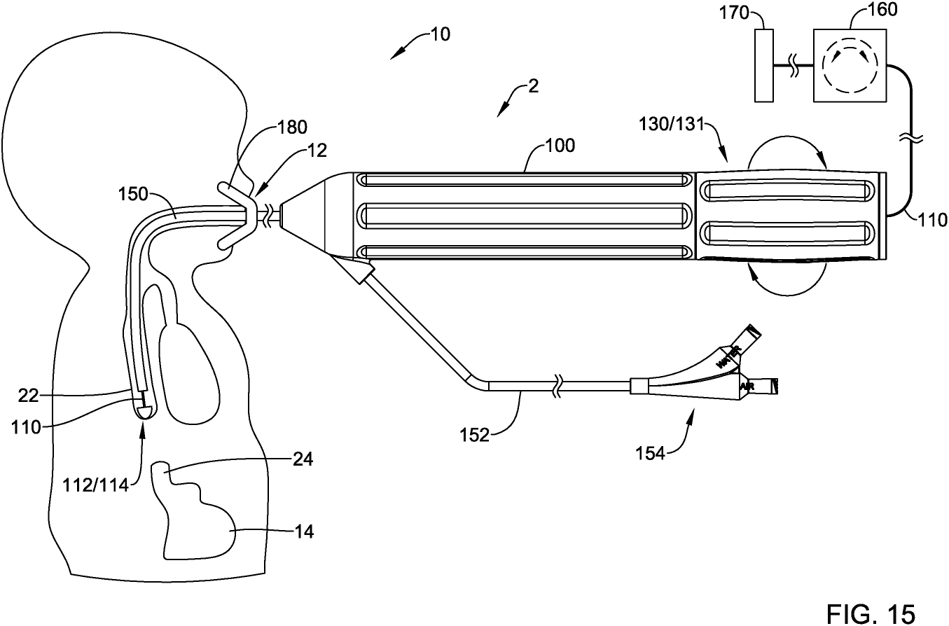

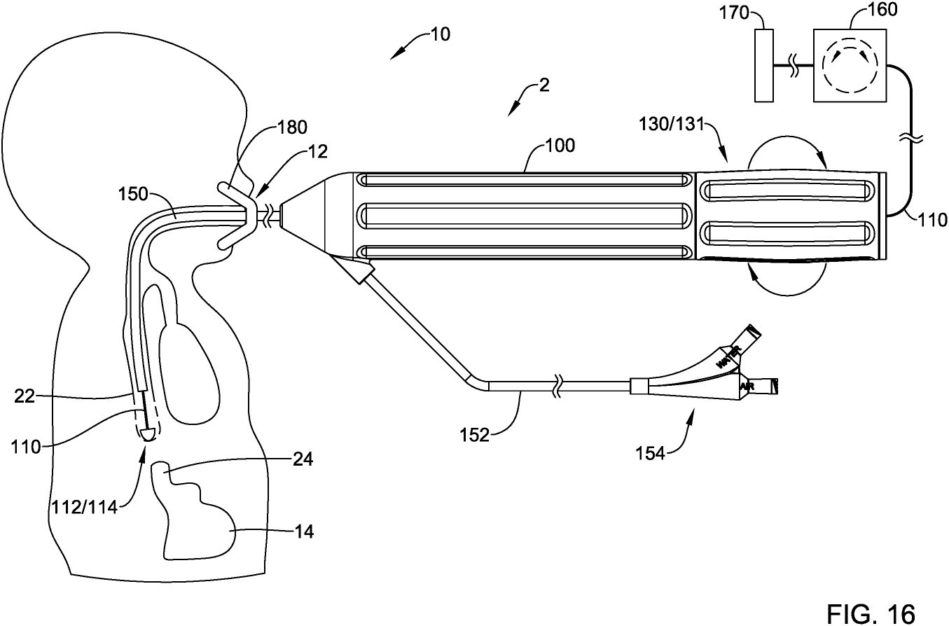

The process described above may be repeated until the elongate shaft 110 and/or the position sensor shaft 144 runs out of available travel within the handle 100. However, the growth and/or stretching process of the upper esophageal pouch 22 of the esophagus 20 may not be complete and/or extensive enough to attach the upper esophageal pouch 22 to the lower esophageal pouch 24 at this time. Accordingly, the medical device 2 may be reset and/or repositioned relative to the patient 10, e.g., the mouth 12, and/or the distal end of the upper esophageal pouch 22, as shown in FIG. 15, and the process may begin again and be repeated as necessary, as shown in FIG. 16. For example, the elongate tubular member 150 and/or the handle 100 may be moved closer to the mouth 12 of the patient, thus moving the distal end 112 and/or atraumatic distal head 114 of the elongate shaft further distally in the upper esophageal pouch 22. Furthermore, the knob 132 may be rotated in the opposite direction to move the first flange 136 proximally within the handle 100 back to or toward an initial position, which also permits the elongate shaft 110 and/or the spring 121 to move proximally relative to the handle 100.

By monitoring and/or tracking the position and/or the location of the elongate shaft 110, the distal end 112 of the elongate shaft 110, and/or the atraumatic distal head 114 relative to the handle 100, the medical device 2 may be precisely repositioned at the distal end of the upper esophageal pouch 22 with knowledge of the current and/or previous displacement of the distal end of the upper esophageal pouch 22 relative to its initial location and/or position. By knowing the change in the length of the upper esophageal pouch 22, an opportunity exists to reduce irradiation and/or imaging of the patient 10 in order to reposition the medical device 2.

In some alternative configurations, the medical device 2 may be used in the lower esophageal pouch 24 in a similar manner. Access to the lower esophageal pouch 24 may be made by a different manner, surgically for example, but the medical device 2 may function in largely the same manner as described herein with respect to the upper esophageal pouch 22.

Once sufficient growth and/or stretch of the distal end of the upper esophageal pouch 22 and/or the lower esophageal pouch 24 (if necessary) has occurred, the upper esophageal pouch 22 and the lower esophageal pouch 24 may be joined together surgically in a known manner to reconnect and/or re-establish the esophagus 20 of the patient 10. The medical device 2 and method described herein may provide numerous benefits over other treatment devices and/or methods. For example, the medical device 2 and method described herein may avoid external suture anchors, surgical access required to make adjustments, induced paralysis of the patient, and/or unintended tissue tearing or rupturing associated with other treatment devices and/or methods while permitting earlier treatment of the patient 10 regardless of patient weight and maintaining adequate suction of and/or within the upper esophageal pouch 22 to prevent mucous and/or saliva from entering the trachea, which could lead to potential disruption of adequate respiration. Other benefits are also contemplated and/or likely to be achieved.

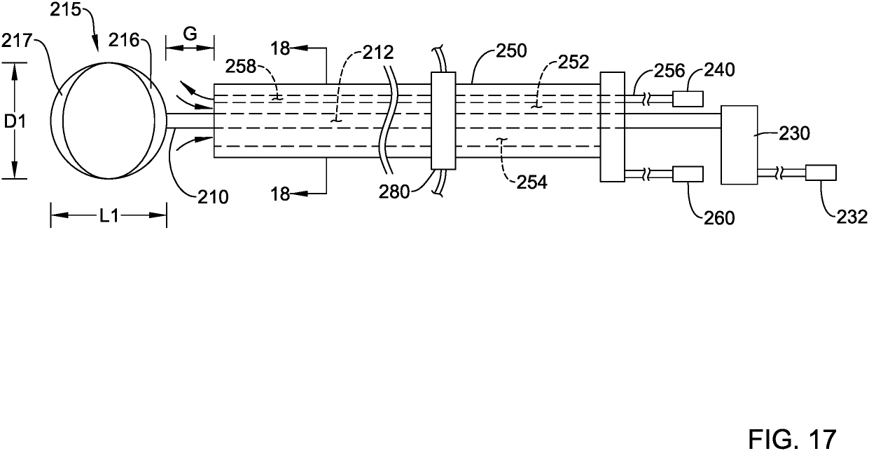

FIGS. 17-19 illustrate an alternative configuration of a medical device 202. In some embodiments, the medical device 202 may optionally include a handle. In some embodiments, the handle may be a generally elongate tubular structure having and/or capable of having elements disposed therein. Other configurations are also contemplated. Some suitable, but non-limiting, examples of materials for the handle, including but not limited to polymeric materials, metallic materials, and/or composite materials, are discussed below.

The medical device 202 may include an elongate tubular member 250 configured to extend through the mouth 12 of the patient 10 and into the upper esophageal pouch 22 of the patient 10, similar to description above with respect to the medical device 2. In some embodiments, the elongate tubular member 250 may be generally flexible in a lateral direction to permit navigation through the mouth 12 of the patient 10 into the esophagus 20 and/or the upper esophageal pouch 22. In some embodiments, the elongate tubular member 250 may be generally rigid and/or stiff in an axial direction to promote pushability and/or transmission of force axially along the elongate tubular member 250. In some embodiments, the elongate tubular member 250 may have a tubular elongate structure having at least one lumen extending therethrough. Other configurations are also contemplated. Some suitable, but non-limiting, examples of materials for the elongate tubular member 250, including but not limited to polymeric materials, metallic materials, and/or composite materials, are discussed below.

The medical device 202 may include an elongate shaft 210 extending distally from the elongate tubular member 250. In some embodiments, the elongate shaft 210 may be generally flexible in a lateral direction to permit navigation through the mouth 12 of the patient 10 into the esophagus 20 and/or the upper esophageal pouch 22. In some embodiments, the elongate shaft 210 may be generally rigid and/or stiff in an axial direction to promote pushability and/or transmission of force axially along the elongate shaft 210. In some embodiments, the elongate shaft 210 may have a tubular elongate structure having at least one lumen extending therethrough. Other configurations are also contemplated. Some suitable, but non-limiting, examples of materials for the elongate shaft 210, including but not limited to polymeric materials, metallic materials, and/or composite materials, are discussed below.

The medical device 202 may include an inflatable member 215 fixedly attached to a distal end of the elongate shaft 210. In some embodiments, the inflatable member 215 may include and/or may be an inflatable balloon. The inflatable member 215 may be configured to shift between a deflated configuration, a partially inflated configuration, and an inflated configuration. The inflatable member 215 may be configured to apply a force to the distal end of the upper esophageal pouch 22 of the patient 10 as the inflatable member 215 is shifted toward the inflated configuration and/or when the inflatable member 215 is in the inflated configuration. Some suitable, but non-limiting, examples of materials for the inflatable member 215, including but not limited to polymeric materials, metallic materials, and/or composite materials, are discussed below.

In some embodiments, the inflatable member 215 may have a first outer diameter D1 in the deflated configuration and/or in the partially inflated configuration, as seen in FIG. 17. The first outer diameter D1 may correspond to a maximum outer extent of the inflatable member 215. In some embodiments, the first outer diameter D1 may be generally constant along a length of the inflatable member 215 such that the inflatable member 215 is generally shaped like a capsule. In some embodiments, the first outer diameter D1 may be about 14 French (Fr) (about 4.67 millimeters (mm)), about 16 Fr (about 5.33 mm), about 18 Fr (about 6.0 mm), about 20 Fr (about 6.67 mm), or another suitable size depending on the size of the patient 10. In one example embodiment, the first outer diameter D1 is about 20 Fr (about 6.67 mm).

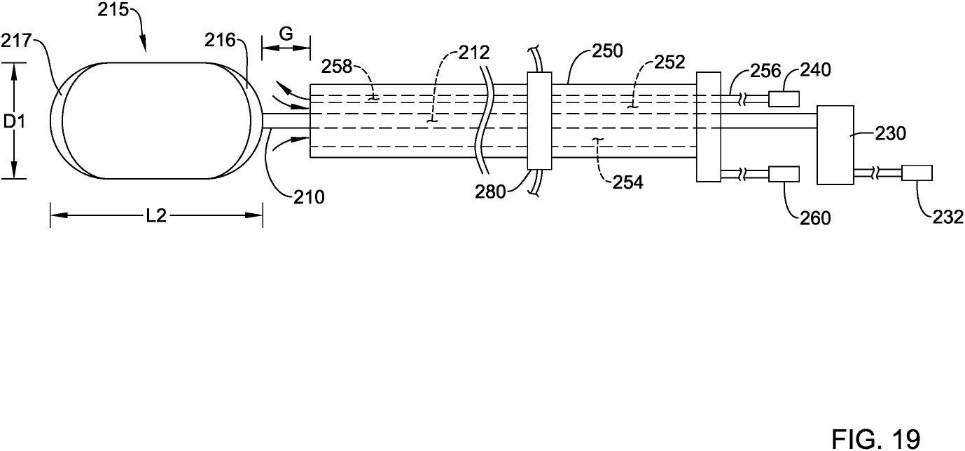

In some embodiments, the inflatable member 215 may have a first length L1 in the deflated configuration and/or in a partially inflated configuration, as seen in FIG. 17. In some embodiments, the inflatable member 215 may be configured to expand in a longitudinal direction to a second length L2 when shifting toward the inflated configuration, as seen in FIG. 19. In some embodiments, the inflatable member 215 may be configured to expand in a longitudinal direction to the second length L2 when shifting from the deflated configuration and/or the partially inflated configuration toward the inflated configuration while maintaining an outer diameter (e.g., the first outer diameter D1) that is substantially constant. In some embodiments, the second length L2 may be about 1 centimeters (cm), about 2 cm, about 1-2 cm, about 3 cm, about 1-3 cm, about 4 cm, about 1-4 cm, about 5 cm. etc. Other configurations are also contemplated.

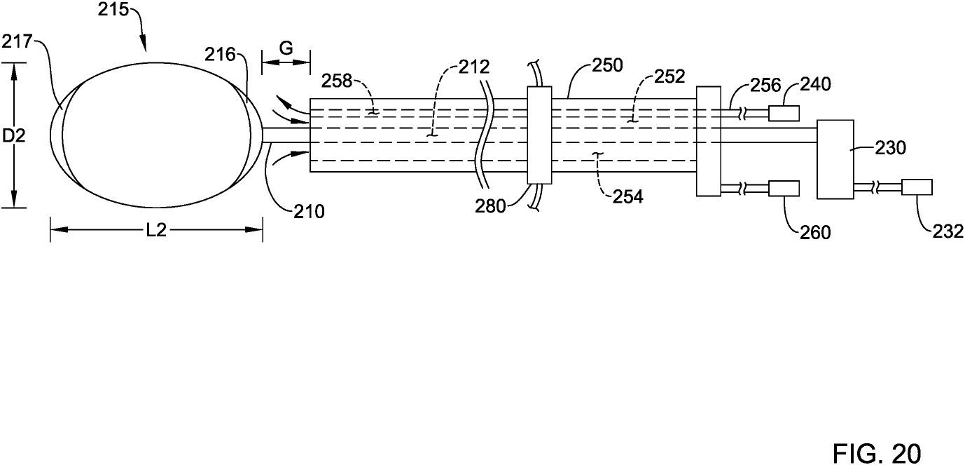

In some alternative embodiments, the first outer diameter D1 may be variable. In some embodiments, the inflatable member 215 may radially expand from the first outer diameter D1 toward and/or to a second outer diameter D2 when shifting from the deflated configuration and/or the partially inflated configuration toward and/or to the inflated configuration, as seen in FIG. 20. Other configurations are also contemplated.

In some alternative embodiments, the inflatable member 215 may include a plurality of folds or a plurality of pleats along its length. In some embodiments, the inflatable member 215 may be configured to expand longitudinally as a bellows or in an accordion-like fashion. Other configurations are also contemplated.

Returning to FIGS. 17-19, in some embodiments, at least a portion of the inflatable member 215 may be radiopaque. In some embodiments, the inflatable member 215 may include a proximal radiopaque portion 216 and/or a distal radiopaque portion 217. In some embodiments, the proximal radiopaque portion 216 and/or the distal radiopaque portion 217 of the inflatable member 215 may include a radiopaque material. In some embodiments, a material forming the inflatable member 215 may be doped with the radiopaque material. In some embodiments, the radiopaque material may be embedded within the material forming the inflatable member 215 to form the proximal radiopaque portion 216 and/or the distal radiopaque portion 217 of the inflatable member 215. In some embodiments, the radiopaque material may be applied to a surface (e.g., an outer surface, an inner surface, etc.) of the inflatable member 215 (e.g., a coating, etc.) to form the proximal radiopaque portion 216 and/or the distal radiopaque portion 217 of the inflatable member 215. In some embodiments, a separate radiopaque member may be fixedly attached to and/or may be bonded to the inflatable member 215 to form the proximal radiopaque portion 216 and/or the distal radiopaque portion 217 of the inflatable member 215. Other configurations are also contemplated.

In some embodiments, the inflatable member 215 may be spaced apart from the distal end of the elongate tubular member 250. In some embodiments, the inflatable member 215 may be spaced apart a fixed distance G from the distal end of the elongate tubular member 250. In at least some embodiments, a proximal end of the inflatable member 215 may be longitudinally fixed and/or immovable relative to the distal end of the elongate tubular member 250. Other configurations are also contemplated.

In some embodiments, the elongate shaft 210 may include an inflation lumen 212 extending therethrough. The inflation lumen 212 may be in fluid communication with the inflatable member 215. In some embodiments, the medical device 202 may further comprise a pressure regulator 230 in fluid communication with the inflation lumen 212 and an inflation fluid source 232. As such, the inflation fluid source 232 may be in fluid communication with the inflation lumen 212 and the inflatable member 215. The inflation fluid source 232 may be adapted to and/or configured to supply inflation fluid to the inflatable member 215 via the inflation lumen 212. In some embodiments, the inflation fluid may include a biocompatible gas. In some embodiments, the inflation fluid may include air. In some embodiments, the inflation fluid may include a biocompatible fluid. In some embodiments, the inflation fluid may include water, saline solution, or another biocompatible fluid or liquid. Other configurations are also contemplated.

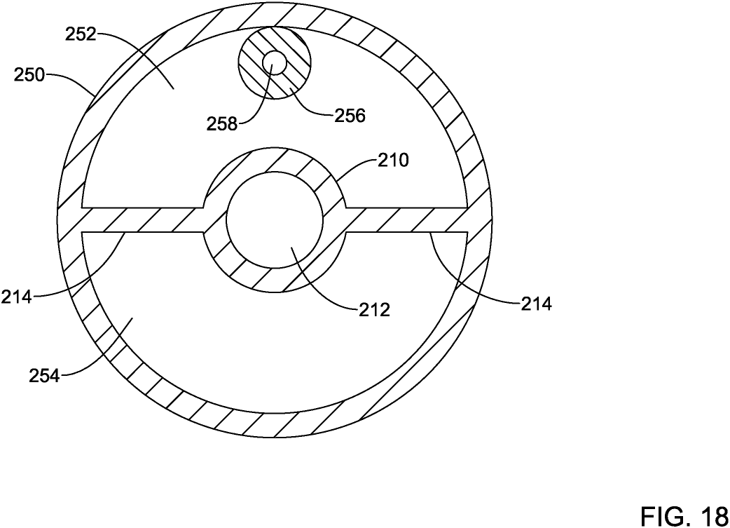

In some embodiments, the elongate tubular member 250 may include an irrigation lumen 258 extending therethrough and at least one suction lumen extending therethrough. In some embodiments, the medical device 202 may include an irrigation tube 256 having the irrigation lumen 258 formed therein extending longitudinally through the at least one suction lumen, as shown in the cross-sectional view of FIG. 18. In some embodiments, the irrigation tube 256 may be formed separately and/or independently from the elongate tubular member 250. As such, in at least some embodiments, the irrigation tube 256 may be removable from the elongate tubular member 250 and/or the at least one suction lumen. Alternatively, in some embodiments, the irrigation tube 256 and/or the irrigation lumen 258 may be integrally and/or monolithically formed with the elongate tubular member 250. Other configurations are also contemplated.

In some embodiments, the irrigation tube 256 and/or the irrigation lumen 258 may be configured to connect to an irrigation fluid source 240 (e.g., water, saline, etc.). In some embodiments, the irrigation tube 256 and/or the irrigation lumen 258 may be used to irrigate the distal end of the upper esophageal pouch 22.

In some embodiments, the at least one suction lumen may be only one lumen. In some embodiments, the at least one suction lumen may include a first suction lumen 252 extending from a proximal end of the elongate tubular member 250 to a distal end of the elongate tubular member 250, and a second suction lumen 254 extending from the proximal end of the elongate tubular member 250 to the distal end of the elongate tubular member 250. In some embodiments, the at least one suction lumen may include more than two suction lumens.

In some embodiments, the at least one suction lumen may be separate and/or independent lumens fluidly isolated from each other within the elongate tubular member 250 (e.g., the first suction lumen 252 and the second suction lumen 254, more than two suction lumens, etc.). In some embodiments, at least one suction lumen may be a single continuous lumen extending from the proximal end of the elongate tubular member 250 to the distal end of the elongate tubular member 250.

In some embodiments, the elongate shaft 210 may be a separate element and/or structure from the elongate tubular member 250 extending longitudinally through the elongate tubular member 250. In some embodiments, the elongate shaft 210 may be integrally and/or monolithically formed with the elongate tubular member 250. In some embodiments, the elongate shaft 210 and/or the elongate tubular member 250 may optionally include a centering structure 214 configured to center, hold, and/or space the elongate shaft 210 radially inward of and/or within the elongate tubular member 250, as seen in FIG. 18. In some embodiments, the centering structure 214 may extend continuously along a length of the elongate shaft 210 and/or the elongate tubular member 250. In one example, the centering structure 214 may separate and/or partially define the first suction lumen 252 and the second suction lumen 254. In some embodiments, the centering structure 214 may extend discontinuously along the length of the elongate shaft 210 and/or the elongate tubular member 250. Other configurations are also contemplated.

In some embodiments, the elongate tubular member 250 may have an outer diameter that is less than the first outer diameter D1. In some embodiments, the outer diameter of the elongate tubular member 250 may be about 6 French (Fr) (about 2.0 millimeters (mm)), about 8 Fr (about 2.67 mm), about 10 Fr (about 3.33 mm), or about 12 Fr (4.0 mm). Other configurations are also contemplated. In some embodiments, the medical device 202 may permit separate support of respiration by other devices alongside and/or while the medical device 202 is in place without impeding respiratory treatment. For example, in some embodiments, the elongate tubular member 250 may be sized to permit intubation alongside the elongate tubular member 250. In some alternative embodiments, the outer diameter of the elongate tubular member 250 may be of sufficient size to accommodate a respiration tube disposed within the elongate tubular member 250.

In some embodiments, the elongate tubular member 250 and/or the at least one suction lumen (e.g., the first suction lumen 252, the second suction lumen 254, etc.) may be configured to connect to a source of suction and/or a vacuum source 260 (e.g., air, etc.). In some embodiments, the source of suction and/or the vacuum source 260 may be configured to suction fluid, mucous, debris, etc. from the distal end of the upper esophageal pouch 22. The at least one suction lumen may have a cross-sectional area and/or a collective cross-sectional area that is greater than a cross-sectional area of the irrigation lumen 258. In at least some embodiments, the medical device 202 may have a greater capacity to suction the distal end of the upper esophageal pouch 22 than to irrigate the distal end of the upper esophageal pouch 22. In some embodiments, the medical device 202 may be configured to treat esophageal atresia and for suction of mucous and/or saliva from the distal end of the upper esophageal pouch 22 simultaneously, thereby preventing mucous and/or saliva from entering the trachea, which could lead to potential disruption of adequate respiration. As such, the medical device 202 is able to help sustain the life of the patient while treating the esophageal atresia.

In some embodiments, the medical device 202 may include an adapter at and/or fixedly attached to the proximal end of the elongate tubular member 250. The adapter may be in fluid communication with the irrigation tube 256 and/or the irrigation lumen 258. The adapter may be in fluid communication with the at least one suction lumen. In some embodiments, the adapter may include a port permitting fluid communication with the elongate shaft 210 and/or the inflation lumen 212. In some embodiments, the adapter may include a port permitting the irrigation tube 256 and/or the irrigation lumen 258 to pass through the adapter and into the elongate tubular member 250. In some embodiments, the adapter may include a port permitting the elongate shaft 210 and/or the inflation lumen 212 to pass through the adapter and into the elongate tubular member 250. Other configurations are also contemplated.

In some embodiments, irrigation of the distal end of the upper esophageal pouch 22 may be periodic and/or performed as needed. In some embodiments, suction of mucous and/or saliva from the distal end of the upper esophageal pouch 22 may be continuous. Other configurations are also contemplated.

In some embodiments, the medical device 202 and/or the elongate tubular member 250 may be secured to the patient 10 and/or may be secured relative to the patient 10, the mouth 12, and/or the distal end of the upper esophageal pouch 22. In some embodiments, the medical device 202 may include and/or may be securable to a fixation element 280. In some embodiments, the fixation element 280 may be secured to the face of the patient 10, such as with adhesive tape. In some embodiments, the fixation element 280 may include a headgear or a mouthpiece secured to the head of the patient 10. In some embodiments, the fixation element 280 may include a mask or other structure configured to at least partially surround and/or encompass the head of the patient 10. In some embodiments, the fixation element 280 may be another medical device or product such as a NeoBar® from Neotech Products. Other suitable medical devices and/or products are also contemplated.

In use, a method of treating esophageal atresia may include inflating and/or expanding the inflatable member 215 to an initial pressure in the partially inflated configuration shown in FIG. 17 prior to insertion into the mouth 12 and/or the upper esophageal pouch 22 of the patient 10. The inflatable member 215 may have and/or be expanded to the first length L1 in the partially inflated configuration.

The method may include inserting the medical device 202 into the mouth 12 and the esophagus 20 of the patient 10. The method may include advancing the medical device 202 toward the upper esophageal pouch 22 until an internal pressure within the inflatable member 215 increases above the initial pressure, thereby indicating that the inflatable member 215 has contacted the distal end of the upper esophageal pouch 22.

In some embodiments, the inflatable member 215 may be configured to engage the distal end of the upper esophageal pouch 22 in a manner that substantially prevents cutting, tearing, abrasion, etc. of the tissue of the upper esophageal pouch 22. In some embodiments, the inflatable member 215 may include a pressure sensor and/or a force sensor disposed therein. In some embodiments, the pressure sensor may be in fluid communication with the inflation lumen 212. In some embodiments, the pressure sensor may be configured to measure the internal pressure within the inflatable member 215 and/or the inflation lumen 212. In some embodiments, the force sensor may be configured to measure a force applied to the distal end of the upper esophageal pouch 22 by the inflatable member 215.

In some embodiments, the pressure sensor and/or the force sensor may include a wire, a fiber optic wire, etc. extending within the elongate shaft 210 to a monitoring device. In some embodiments, the monitoring device may include a display indicating a value of the force and/or a pressure applied to esophageal tissue by the inflatable member 215. In some embodiments, the monitoring device may include and/or emit visual and/or audible alarms if the force exceeds a predetermined maximum force and/or if the force drops below a predetermined force threshold. In some embodiments, the monitoring device may include and/or emit visual and/or audible alarms if the pressure exceeds a predetermined maximum pressure and/or if the pressure drops below a predetermined pressure threshold. In some embodiments, the monitoring device may include and/or may be in electronic communication with a computer, a microprocessor, a network, a cloud computing service, or other communications equipment.

In some embodiments, the method may include securing the medical device 202 and/or the elongate tubular member 250 relative to the patient 10. In some embodiments, the elongate tubular member 250 may be fixed to the fixation element 280, which may then the secured to and/or relative to the patient 10 and/or the mouth 12 of the patient 10. In some embodiments, the fixation element 280 may be secured to the face of the patient 10, such as with adhesive tape. In some embodiments, the fixation element 280 may include a headgear or a mouthpiece secured to the head of the patient 10. In some embodiments, the fixation element 280 may include a mask or other structure configured to at least partially surround and/or encompass the head of the patient 10. In some embodiments, the medical device 202 and/or the elongate tubular member 250 may be secured to the patient 10 and/or may be secured relative to the patient 10, the mouth 12, and/or the distal end of the upper esophageal pouch 22 by securing the medical device 202 and/or the elongate tubular member 250 to another medical device or product such as a NeoBar® from Neotech Products. Other suitable medical devices and/or products are also contemplated.

After securing the medical device 202 and/or the elongate tubular member 250 relative to the patient 10, the method may include setting a desired pressure setting on and/or within the pressure regulator 230. The pressure regulator 230 may be responsive to sensed pressure (e.g., the internal pressure) within the inflatable member 215 and/or the inflation lumen 212. The pressure regulator 230 may be configured to inflate the inflatable member 215 to the desired pressure setting automatically or upon being instructed or commanded by the user or a control system (e.g., the monitoring device, etc.). In some embodiments, the desired pressure setting may be higher than the initial pressure. In some embodiments, the desired pressure setting must be higher than the initial pressure. In some alternative embodiments, the desired pressure setting may be the same as the initial pressure. As the internal pressure in the inflatable member 215 increases, the inflatable member 215 may expand longitudinally and/or the inflatable member 215 may apply force to the distal end of the upper esophageal pouch 22 of the patient 10. The force sensor may be configured to measure the force applied to the distal end of the upper esophageal pouch 22 of the patient 10.

In some embodiments, the force may be applied constantly to the distal end of the upper esophageal pouch 22 of the patient 10. In some embodiments, the force may be varied over time. In some embodiments, the force may be applied cyclically to the distal end of the upper esophageal pouch 22 of the patient 10. For example, in some embodiments, the force may be applied to the distal end of the upper esophageal pouch 22 of the patient 10 for a first interval of time and the force may be removed for a second interval of time, wherein the first interval of time alternates with the second interval of time. In some embodiments, the force may be applied to the distal end of the upper esophageal pouch 22 of the patient 10 at an original value until a certain amount of growth of the upper esophageal pouch 22 occurs and/or the force drops below a certain amount from the original value, and then the force may be increased and/or re-applied back to the original value. In some embodiments, the force may be applied to the distal end of the upper esophageal pouch 22 of the patient 10 such that the force follows a cyclical wave (e.g., a sine wave, etc.), wherein the force is slowly increased over time and then slow decreased over time in a repeating fashion and/or pattern. Other configurations are also contemplated. In some embodiments, the cyclical application of force may allow and/or permit at least some tissue relaxation and/or reperfusion during the duration of process and/or procedure. In some embodiments, force may be applied randomly to the distal end of the upper esophageal pouch 22 of the patient 10 as the patient 10 moves. In some embodiments, the force may vary randomly about a target force value (e.g., the original force). In some embodiments, the force may be applied randomly to the distal end of the upper esophageal pouch 22 of the patient 10 about the target force value with a preset and/or user set maximum force value. Other configurations, including various combinations thereof, are also contemplated. In some embodiments, the force may be no more than 1.0 pounds of force. In some embodiments, the force may be no more than 0.8 pounds of force. In some embodiments, the force may be no more than 0.6 pounds of force. In some embodiments, the force may be no more than 0.5 pounds of force. Other configurations are also contemplated.

After setting the desired pressure setting, the method may include setting a maximum pressure limit on and/or within the pressure regulator 230. In some embodiments, the maximum pressure limit may be higher than the desired pressure setting. In some embodiments, the maximum pressure limit must be higher than the desired pressure setting. In some alternative configurations, the maximum pressure limit may be the same as the desired pressure setting.

The method may also include setting a minimum pressure limit on and/or within the pressure regulator 230. In some embodiments, when the internal pressure within the inflatable member 215 falls to or below the minimum pressure limit, the inflatable member 215 may be re-inflated and/or re-pressurized to the desired pressure setting.

In some embodiments, the inflatable member 215 may act as a force absorbing member. In some embodiments, the inflatable member 215 may be configured to deflate and/or to shift toward and/or to the partially inflated configuration (e.g., the initial pressure) in response to movement(s) of the patient 10 and/or an increase in the internal pressure within the inflatable member 215 to and/or above the maximum pressure limit. In some alternative embodiments, the inflatable member 215 may be configured to deflate and/or to shift toward the deflated configuration (e.g., zero pressure) in response to movement(s) of the patient 10 and/or an increase in the internal pressure within the inflatable member 215 to and/or above the maximum pressure limit. In some embodiments, the inflatable member 215 may be configured to deflate and/or to shift toward and/or to the partially inflated configuration (e.g., the initial pressure) in response to movement(s) of the patient 10 and/or an increase in the force exerted upon the distal end of the upper esophageal pouch as measured by the force sensor above a preselected or user set maximum force.

As the patient 10 moves, the internal pressure within the inflatable member 215 may change. Within a predetermined range from the desired pressure setting, no changes to the internal pressure are made. In some embodiments, the predetermined range may be +/- 5 mmH2O. In some embodiments, the predetermined range may be +/- 10 mmH2O. Other configurations are also contemplated.

In some embodiments, the inflatable member 215 may be configured to inflate and/or deflate in response to movements of the patient 10 that change the internal pressure within the inflatable member 215 to a value outside of the predetermined range from the desired pressure setting. As the patient 10 moves due to respiration, coughing, sneezing, crying, etc. the inflatable member 215 may deflate and inflate in an oscillatory manner (e.g., back and forth in a longitudinal direction), thereby reducing the potential and/or opportunity for rupture for the upper esophageal pouch 22. In some embodiments, the oscillatory deflation and inflation of the inflatable member 215 may also create and/or permit some occasional variation in the force applied to the distal end of the upper esophageal pouch 22. Such variation may permit and/or encourage reperfusion of the tissue at the distal end of the upper esophageal pouch 22, thereby reducing tissue morbidity and/or necrosis while providing sufficient force to induce growth and/or stretch of the distal end of the upper esophageal pouch 22 over time.

As the distal end of the upper esophageal pouch 22 grows and/or stretches, the internal pressure within the inflatable member 215 will decrease. If and/or when the internal pressure within the inflatable member 215 falls to or reaches the minimum pressure limit, the inflatable member 215 may be re-inflated to the desired pressure setting. This process may continue until imaging and/or pressure readings indicate that the inflatable member 215 has reached its maximum longitudinal length. At this point, the medical device 202 and/or the elongate tubular member 250 may be released from the patient 10 and/or the fixation element 280, reset to the initial pressure within the inflatable member 215, repositioned against the distal end of the upper esophageal pouch 22 as described above, and then resecured relative to the patient 10 and/or the fixation element 280.

In some embodiments, the inflatable member 215 may be configured to apply a force to the distal end of the upper esophageal pouch 22 of the patient 10. In some embodiments, the force sensor disposed within the inflatable member 215 may be configured to measure the force applied to the distal end of the upper esophageal pouch 22 by the inflatable member 215. In some embodiments, the inflatable member 215 may be configured to inflate and/or deflate while maintaining the force against the distal end of the upper esophageal pouch 22.

In some embodiments, the method may include monitoring the force applied to the distal end of the upper esophageal pouch 22 over time. In some embodiments, the method may include monitoring pressure within the inflatable member 215 over time. In some embodiments, the method may include increasing the pressure within the inflatable member 215 to increase the force applied to the distal end of the upper esophageal pouch 22 when the force drops below the predetermined force threshold and/or when the pressure drops below the minimum pressure limit.

In some embodiments, the predetermined force threshold may be 0.5 pounds of force. In some embodiments, the predetermined force threshold may be 0.4 pounds of force. In some embodiments, the predetermined force threshold may be 0.3 pounds of force. In some embodiments, the predetermined force threshold may be 0.2 pounds of force. In some embodiments, the predetermined force threshold may be 0.1 pounds of force. Other configurations and/or values are also contemplated.

In one nonlimiting example, the inflatable member 215 may be inflated to an initial pressure of about 20 mmH2O (e.g., the internal pressure). As the medical device 202 is inserted into the upper esophageal pouch 22, the internal pressure will begin to rise when the distal end of the inflatable member 215 presses against the distal end of the upper esophageal pouch 22. At this point, the medical device 202 and/or the elongate tubular member 250 may be secured in place relative to the patient 10 and/or the distal end of the upper esophageal pouch 22. The pressure regulator 230 may be set to the desired pressure setting – 50 mmH2O, for example. As the patient 10 moves, the internal pressure within the inflatable member 215 may change. Within a predetermined range from the desired pressure setting, no changes to the internal pressure are made. For example, the predetermined range may be +/- 10 mmH2O. When the pressure sensor detects the internal pressure within the inflatable member 215 has risen to above 60 mmH2O, the pressure regulator 230 may deflate the inflatable member 215 toward and/or to the partially inflated configuration (e.g., the initial pressure, or 20 mmH2O). Alternatively, when the pressure sensor detects the internal pressure within the inflatable member 215 has risen to above 60 mmH2O, the pressure regulator 230 may deflate the inflatable member 215 toward and/or to the minimum pressure limit (e.g., 30-40 mmH2O). When the minimum pressure limit is reached and/or when the initial pressure is reached, the pressure regulator 230 may reinflate the inflatable member 215 to the desired pressure setting (e.g., 50 mmH2O). In some embodiments, the pressure regulator 230 may incorporate a time delay between deflation and inflation to ensure that movements of the patient have passed.

In some alternative configurations, the medical device 202 may be used in the lower esophageal pouch 24 in a similar manner. Access to the lower esophageal pouch 24 may be made by a different manner, surgically for example, but the medical device 202 may function in largely the same manner as described herein with respect to the upper esophageal pouch 22.

Once sufficient growth and/or stretch of the distal end of the upper esophageal pouch 22 and/or the lower esophageal pouch 24 (if necessary) has occurred, the upper esophageal pouch 22 and the lower esophageal pouch 24 may be joined together surgically in a known manner to reconnect and/or re-establish the esophagus 20 of the patient 10. The medical device 202 and method described herein may provide numerous benefits over other treatment devices and/or methods. For example, the medical device 202 and method described herein may avoid external suture anchors, surgical access required to make adjustments, induced paralysis of the patient, and/or unintended tissue tearing or rupturing associated with other treatment devices and/or methods while permitting earlier treatment of the patient 10 regardless of patient weight. Other benefits are also contemplated and/or likely to be achieved.

The materials that can be used for the various components of the medical device 2 and the various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the handle, the elongate shaft, the elongate tubular member, the force absorbing member, the spring, the adjustment mechanism, the knob, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In some embodiments, a linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol, can be used to achieve desired properties.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system and/or components thereof in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system and/or components thereof to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (i.e., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anticoagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made to details, particularly in matters of shape, size, and arrangement of steps without exceeding the scope of the disclosure. This may include, to the extent that it is appropriate, the use of any of the features of one example embodiment being used in other embodiments. The disclosure’s scope is, of course, defined in the language in which the appended claims are expressed.

What is claimed is:

1. A medical device for treating esophageal atresia, comprising:
a handle;
an elongate shaft extending distally from the handle, the elongate shaft configured to extend through a mouth of a patient and into an upper esophageal pouch of the patient, the elongate shaft having a distal end configured to engage a distal end of the upper esophageal pouch; and
a force absorbing member disposed within the handle, the force absorbing member permitting the elongate shaft to translate axially relative to the handle, wherein the force absorbing member is configured to exert a force along the elongate shaft to apply the force to the distal end of the upper esophageal pouch.

2. The medical device of claim 1, further comprising an adjustment mechanism configured to adjust the force applied to the distal end of the upper esophageal pouch by the force absorbing member.

3. The medical device of claim 2, wherein the adjustment mechanism is configured to axially translate the force absorbing member within the handle.

4. The medical device of claim 1, further comprising a position sensor disposed within the handle, the position sensor being configured to detect an axial position of the elongate shaft relative to the handle.

5. The medical device of claim 1, wherein the elongate shaft is fixedly secured relative to a distal end of the force absorbing member.

6. The medical device of claim 1, wherein the elongate shaft extends axially through the handle and engages with a longitudinal advancement-retraction mechanism disposed proximal of the handle.

7. The medical device of claim 1, wherein the force absorbing member is a spring.

8. The medical device of claim 1, wherein the adjustment mechanism includes:
a knob disposed proximate a proximal end of the handle; and
a threaded shaft extending distally from the knob within the handle;
wherein a distal portion of the threaded shaft includes a first flange abutting a proximal end of the spring and rotation of the knob relative to the handle translates the first flange axially within the handle.

9. The medical device of claim 8, wherein the elongate shaft extends axially through the knob and is movable with respect to the knob.

10. A medical device for treating esophageal atresia, comprising:
an elongate tubular member configured to extend through a mouth of a patient and into an upper esophageal pouch of the patient;
an elongate shaft extending distally from the elongate tubular member; and
an inflatable member fixedly attached to a distal end of the elongate shaft, the inflatable member being configured to shift between a deflated configuration and an inflated configuration;
wherein the inflatable member is configured to apply a force to a distal end of the upper esophageal pouch in the inflated configuration;
wherein the elongate shaft includes an inflation lumen in fluid communication with the inflatable member;
wherein the elongate tubular member includes an irrigation lumen extending therethrough and at least one suction lumen extending therethrough.

11. The medical device of claim 10, wherein the inflatable member is spaced apart a fixed distance from a distal end of the elongate tubular member.

12. The medical device of claim 10, wherein the inflatable member is configured to expand in a longitudinal direction when shifting toward the inflated configuration while maintaining an outer diameter that is substantially constant.

13. The medical device of claim 10, further comprising a pressure regulator in fluid communication with the inflation lumen, the pressure regulator being responsive to sensed pressure within the inflatable member.

14. The medical device of claim 10, wherein at least a portion of the inflatable member is radiopaque.

15. A method of treating esophageal atresia, comprising:
inserting a medical device for treating esophageal atresia into a mouth and esophagus of a patient, the medical device comprising:
a handle including an adjustment mechanism;
an elongate shaft extending distally from the handle, the elongate shaft having a distal end configured to engage a distal end of an upper esophageal pouch of the patient; and
a force absorbing member disposed within the handle and configured to exert a force along the elongate shaft to apply the force to the distal end of the upper esophageal pouch;
positioning the distal end of the elongate shaft against the distal end of the upper esophageal pouch;
fixing the medical device in place relative to the distal end of the upper esophageal pouch;
actuating the adjustment mechanism to adjust the force applied to the distal end of the upper esophageal pouch by the force absorbing member;
wherein the force absorbing member is configured to permit the elongate shaft to translate axially relative to the handle while maintaining the force against the distal end of the upper esophageal pouch.

16. The method of claim 15, wherein the force is applied constantly to the distal end of the upper esophageal pouch.

17. The method of claim 15, wherein the force is applied cyclically the distal end of the upper esophageal pouch.

18. The method of claim 15, wherein the force is no more than 0.8 pounds of force.

19. The method of claim 15, further comprising:
monitoring the force applied to the distal end of the upper esophageal pouch over time;
monitoring axial translation of the elongate shaft over time; and
actuating the adjustment mechanism to increase the force when the force drops below a predetermined force threshold or when axial translation of the elongate shaft reaches a predetermined translation threshold.

20. The method of claim 15, wherein a pressure sensor is disposed within the distal end of the elongate shaft, the pressure sensor being configured to measure the force applied to the distal end of the upper esophageal pouch.
